(19) United States
(12) Patent Application Publication
(10) Pub. No.: US 2022/0104532 A1
(43) Pub. Date: Apr. 7, 2022

(54) METHODS OF MAKING TOBACCO-FREE SUBSTRATES FOR AEROSOL DELIVERY DEVICES

(71) Applicant: NIlCOVENTURES TRADING LIMITED, London (GB)

(72) Inventors: John Paul Mua, Advance, NC (US); Luis R. Monsalud, Kernersville, NC (US); Andries Don Sebastian, Winston Salem, NC (US); Thaddeus Jackson, Summerfield, NC (US)

(21) Appl. No.: 17/065,159

(22) Filed: Oct. 7, 2020

(51) Int. Cl. A24B 15/167; A24B 15/40; A24F 40/20; A24D 1/22

(52) U.S. Cl. CPC A24B 15/167; A24B 15/40; A61M 15/06; A24D 1/22; A24F 40/20

(57) ABSTRACT

The present disclosure provides an aerosol generating component containing a substrate carrying at least one aerosol forming material. The substrate is substantially free of solid tobacco material, and includes about 60% dry weight or more of one or more fillers, based on the total dry weight of the substrate, and at least one binder. The final form of the substrate can be configured to be used in aerosol generating components for aerosol delivery devices. Such devices utilize electrically generated heat or combustible ignition sources to heat the aerosol forming materials, providing an inhalable substance in the form of an aerosol.

### FIELD OF THE DISCLOSURE

The present disclosure relates to aerosol generating components, aerosol delivery devices, and aerosol delivery systems, such as smoking articles that utilize electrically-generated heat or combustible ignition sources to heat aerosol forming materials, generally without significant combustion, in order to provide an inhalable substance in the form of an aerosol for human consumption.

### BACKGROUND

Many smoking articles have been proposed through the years as improvements upon, or alternatives to, smoking products based upon combusting tobacco for use. Some example alternatives have included devices wherein a solid or liquid fuel is combusted to transfer heat to tobacco or wherein a chemical reaction is used to provide such heat source. Additional example alternatives use electrical energy to heat tobacco and/or other aerosol generating substrate materials, such as described in U.S. Patent No. 9,078,473 to Worm et al., which is incorporated herein by reference in its entirety.

The point of the improvements or alternatives to smoking articles typically has been to provide the sensations associated with cigarette, cigar, or pipe smoking, without delivering considerable quantities of incomplete combustion and pyrolysis products. To this end, there have been proposed numerous smoking products, flavor generators, and medicinal inhalers which utilize electrical energy to vaporize or heat a volatile material, or attempt to provide the sensations of cigarette, cigar, or pipe smoking without burning tobacco to a significant degree. See, for example, the various alternative smoking articles, aerosol delivery devices and heat generating sources set forth in the background art described in U.S. Pat. No. 7,726,320 to Robinson et al.; and U.S. Pat. App. Pub. Nos. 2013/0255702 to Griffith, Jr. et al.; and 2014/0096781 to Sears et al., each of which are incorporated herein by reference in their entireties.

Articles that produce the taste and sensation of smoking by electrically heating tobacco, tobacco-derived materials, or other plant derived materials have suffered from inconsistent performance characteristics. For example, some articles have suffered from inconsistent release of flavors or other inhalable materials, inadequate loading of aerosol forming materials on substrates, or the presence of poor sensory characteristics. Accordingly, it can be desirable to provide a smoking article that can provide the sensations of cigarette, cigar, or pipe smoking, that does so without combusting the substrate material and that does so with advantageous performance characteristics.

### BRIEF SUMMARY

The present disclosure relates to aerosol generating components and aerosol delivery devices that utilize electrically-generated heat or combustible ignition sources to heat a substrate such as a tobacco-free HNB substrate for aerosol delivery devices, in order to provide an inhalable substance in the form of an aerosol for human consumption.

Accordingly, in one aspect, the disclosure provides an aerosol generating component comprising a substrate carrying at least one aerosol forming material, the substrate comprising: about 60% dry weight or more of one or more fillers, based on the total dry weight of the substrate, wherein calcium carbonate comprises at least about 80% by dry weight of the one or more fillers; and at least one binder at least one binder selected from the group consisting of alginate salts, seaweed hydrocolloids, carrageenans, agar, hydroxyalkyl cellulose ethers, and combinations thereof; and wherein the substrate is substantially free of solid tobacco material.

In some embodiments, the substrate comprises at least about 70% by dry weight of the one or more fillers, based on the total dry weight of the substrate. In some embodiments, the substrate comprises at least about 75% by dry weight of the one or more fillers, based on the total dry weight of the substrate.

In some embodiments, the substrate comprises at least about 55% by dry weight calcium carbonate, based on the total dry weight of the substrate. In some embodiments, the substrate comprises at least about 60% by dry weight calcium carbonate, based on the total dry weight of the substrate.

In some embodiments, the substrate is substantially free of rice starch and rice flour.

In some embodiments, the substrate is substantially free of wood fibers.

In some embodiments, the one or more fillers further comprise native or modified starches, maltodextrin, dextrose, calcium phosphate, lactose, sugar alcohols, microcrystalline cellulose, or a combination thereof.

In some embodiments, the at least one binder is selected from the group consisting of the at least one binder is selected from the group consisting of alginate salts, seaweed hydrocolloids, carrageenans, agar, and combinations thereof. In some embodiments, the at least one binder is a hydroxyalkyl cellulose ether selected from the group consisting of methylcellulose, hydroxypropyl cellulose (HPC), hydroxypropyl methylcellulose (HPMC), hydroxyethyl cellulose, and combinations thereof. In some embodiments, the at least one binder is selected from the group consisting of hydroxypropyl cellulose (HPC), hydroxypropyl methylcellulose (HPMC), and combinations thereof. In some embodiments, the at least one binder comprises a combination of HPC and HPMC. In some embodiments, a weight ratio of HPC to HPMC is at least about 1:1, such as about 1:1 to about 5:1, about 2:1 to about 4:1, or about 3:1.

In some embodiments, the at least one aerosol forming material comprises water, a polyhydric alcohol, pa olysorbate, a sorbitan ester, a fatty acid, a fatty acid ester, a wax, a cannabinoid, a terpene, a sugar alcohol, or combinations of any thereof. In some embodiments, the at least one aerosol forming material comprises a polyhydric alcohol. In some embodiments, the polyhydric alcohol is selected from the group consisting of glycerol, propylene glycol, 1,3-propanediol, diethylene glycol, triethylene glycol, triacetin, and combinations thereof.

In some embodiments, the substrate carries the at least one aerosol forming material at a loading of from about 15 to about 55% by weight, based on the total weight of the substrate and aerosol forming material.

In some embodiments, the substrate further carries a flavorant, an active ingredient, or a combination thereof. In some embodiments, the active ingredient comprises a nicotine component.

In some embodiments, the substrate is in particulate form, shredded form, film form, paper process sheet form, cast sheet form, bead form, granular rod form, or extrudate form. In some embodiments, the substrate is formed into a substantially cylindrical shape, the substrate optionally including at least one of an orifice extending through the substantially cylindrical shape and one or more grooves in an exterior surface thereof.

In another aspect is provided an aerosol generating component comprising a substrate carrying at least one aerosol forming material, the substrate comprising: about 60% dry weight or more of one or more fillers, based on the total dry weight of the substrate; and at least one binder, the at least one binder comprising a combination of hydroxypropyl cellulose (HPC) and hydroxypropyl methylcellulose (HPMC); wherein the substrate is substantially free of solid tobacco material.

In some embodiments, a weight ratio of HPC to HPMC is at least about 1:1, such as about 1:1 to about 5:1 or about 2:1 to about 4:1 or about 3:1.

In some embodiments, the one or more fillers comprises native or modified starches, maltodextrin, dextrose, calcium phosphate, lactose, sugar alcohols, microcrystalline cellulose, or a combination thereof.

In yet another aspect is provided an aerosol delivery device, comprising: the aerosol generating component as disclosed herein; a heat source configured to heat the substrate carrying the one or more aerosol forming materials to form an aerosol; and an aerosol pathway extending from the aerosol generating component to a mouth-end of the aerosol delivery device.

In some embodiments, the heat source comprises either an electrically powered heating element or a combustible ignition source.

In some embodiments, the heat source is a combustible ignition source comprising a carbon-based material.

In some embodiments, the heat source is an electrically-powered heating element. In some embodiments, the aerosol delivery device further comprises a power source electronically connected to the heating element. In some embodiments, the aerosol delivery device further comprises a controller configured to control the power transmitted by the power source to the heating element.

The disclosure includes, without limitations, the following embodiments.

Embodiment 1: An aerosol generating component comprising a substrate carrying at least one aerosol forming material, the substrate comprising: about 60% dry weight or more of one or more fillers, based on the total dry weight of the substrate, wherein calcium carbonate comprises at least about 80% by dry weight of the one or more fillers; and at least one binder at least one binder selected from the group consisting of alginate salts, seaweed hydrocolloids, carrageenans, agar, hydroxyalkyl cellulose ethers, and combinations thereof; and wherein the substrate is substantially free of solid tobacco material.

Embodiment 2: The aerosol generating component of embodiment 1, wherein the substrate comprises at least about 70% by dry weight of the one or more fillers, based on the total dry weight of the substrate.

Embodiment 3: The aerosol generating component of embodiment 1 or 2, wherein the substrate comprises at least about 75% by dry weight of the one or more fillers, based on the total dry weight of the substrate.

Embodiment 4: The aerosol generating component of any one of embodiments 1 to 3, wherein the substrate comprises at least about 55% by dry weight calcium carbonate, based on the total dry weight of the substrate, based on the total dry weight of the substrate.

Embodiment 5: The aerosol generating component of any one of embodiments 1 to 4, wherein the substrate comprises at least about 60% by dry weight calcium carbonate, based on the total dry weight of the substrate.

Embodiment 6: The aerosol generating component of any one of embodiments 1 to 5, wherein the substrate is substantially free of rice starch.

Embodiment 7: The aerosol generating component of any one of embodiments 1 to 6, wherein the substrate is substantially free of wood fibers.

Embodiment 8: The aerosol generating component of any one of embodiments 1 to 7, wherein the one or more fillers further comprise maltodextrin, dextrose, calcium phosphate, lactose, sugar alcohols, microcrystalline cellulose, or a combination thereof.

Embodiment 9: The aerosol generating component of any one of embodiments 1 to 8, wherein the at least one binder is selected from the group consisting of alginate salts, seaweed hydrocolloids, carrageenans, agar, and combinations thereof.

Embodiment 10: The aerosol generating component of any one of embodiments 1 to 9, wherein the at least one binder is a hydroxyalkyl cellulose ether selected from the group consisting of methylcellulose, hydroxypropyl cellulose (HPC), hydroxypropyl methylcellulose (HPMC), hydroxyethyl cellulose, and combinations thereof.

Embodiment 11: The aerosol generating component of any one of embodiments 1 to 10, wherein the at least one binder is selected from the group consisting of hydroxypropyl cellulose (HPC), hydroxypropyl methylcellulose (HPMC), and combinations thereof.

Embodiment 12: The aerosol generating component of any one of embodiments 1 to 11, wherein the at least one binder comprises a combination of HPC and HPMC.

Embodiment 13: The aerosol generating component of any one of embodiments 1 to 12, wherein a weight ratio of HPC to HPMC is at least about 1:1, such as about 1:1 to about 5:1, about 2:1 to about 4:1, or about 3:1.

Embodiment 14: The aerosol generating component of any one of embodiments 1 to 13, wherein the at least one aerosol forming material comprises water, a polyhydric alcohol, a polysorbate, a sorbitan ester, a fatty acid, a fatty acid ester, a wax, a cannabinoid, s terpene, a sugar alcohol, or a combination of any thereof.

Embodiment 15: The aerosol generating component of any one of embodiments 1 to 14, wherein the at least one aerosol forming material comprises a polyhydric alcohol.

Embodiment 16: The aerosol generating component of any one of embodiments 1 to 15, wherein the polyhydric alcohol is selected from the group consisting of glycerol, propylene glycol, 1,3-propanediol, diethylene glycol, triethylene glycol, triacetin, and combinations thereof.

Embodiment 17: The aerosol generating component of any one of embodiments 1 to 16, wherein the substrate carries the at least one aerosol forming material at a loading of from about 15 to about 55% by weight, based on the total weight of the substrate and aerosol forming material.

Embodiment 18: The aerosol generating component of any one of embodiments 1 to 17, wherein the substrate further carries a flavorant, an active ingredient, or a combination thereof.

Embodiment 19: The aerosol generating component of any one of embodiments 1 to 18, wherein the active ingredient comprises a nicotine component.

Embodiment 20: The aerosol generating component of any one of embodiments 1 to 19, wherein the substrate is in particulate form, shredded form, film form, paper process sheet form, cast sheet form, bead form, granular rod form, or extrudate form.

Embodiment 21: The aerosol generating component of any one of embodiments 1 to 20, wherein the substrate is formed into a substantially cylindrical shape, the substrate optionally including at least one of an orifice extending through the substantially cylindrical shape and one or more grooves in an exterior surface thereof.

Embodiment 22: An aerosol generating component comprising a substrate carrying at least one aerosol forming material, the substrate comprising: about 60% dry weight or more of one or more fillers, based on the total dry weight of the substrate; and at least one binder, the at least one binder comprising a combination of hydroxypropyl cellulose (HPC) and hydroxypropyl methylcellulose (HPMC); wherein the substrate is substantially free of solid tobacco material.

Embodiment 23: The aerosol generating component of embodiment 22, wherein a weight ratio of HPC to HPMC is at least about 1:1, such as about 1:1 to about 5:1 or about 2:1 to about 4:1 or about 3:1.

Embodiment 24: The aerosol generating component of any one of embodiments 22 to 23, wherein the one or more fillers comprises maltodextrin, dextrose, calcium phosphate, lactose, sugar alcohols, microcrystalline cellulose, or a combination thereof.

Embodiment 25: An aerosol delivery device comprising: the aerosol generating component of any one of embodiments 1 to 24; a heat source configured to heat the substrate carrying the one or more aerosol forming materials to form an aerosol; and an aerosol pathway extending from the aerosol generating component to a mouth-end of the aerosol delivery device.

Embodiment 26: The aerosol delivery device of embodiment 25, wherein the heat source comprises either an electrically powered heating element or a combustible ignition source.

Embodiment 27: The aerosol delivery device of embodiment 25, wherein the heat source is a combustible ignition source comprising a carbon-based material.

Embodiment 28: The aerosol delivery device of any one of embodiments 25 to 27, wherein the heat source is an electrically-powered heating element.

Embodiment 29: The aerosol delivery device of any one of embodiments 25 to 28, further comprising a power source electronically connected to the heating element.

Embodiment 30: The aerosol delivery device of any one of embodiments 25 to 29, further comprising a controller configured to control the power transmitted by the power source to the heating element.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are exemplary only, and should not be construed as limiting the disclosure.

FIG. 1 illustrates a perspective view of an aerosol delivery device comprising a control body and an aerosol generating component, wherein the aerosol generating component and the control body are coupled to one another, according to an example embodiment of the present disclosure;

FIG. 2 illustrates a perspective view of the aerosol delivery device of FIG. 1 wherein the aerosol generating component and the control body are decoupled from one another, according to an example embodiment of the present disclosure;

FIG. 3 illustrates a perspective schematic view of an aerosol generating component, according to an example embodiment of the disclosure;

FIG. 4 illustrates a schematic cross-section drawing of a substrate portion of an aerosol generating component, according to an example embodiment of the present disclosure;

FIGS. 5A, 5B, 5C, and 5D illustrate perspective views of a substrate portion of an aerosol generating component in which the substrate is extruded and in substantially a cylindrical shape (FIGS. 5A-5C), or is cast or extruded as a flat sheet (FIG. 5D);

FIG. 6 illustrates a perspective view of an aerosol generating component, according to an example embodiment of the present disclosure;

FIG. 7 illustrates a perspective view of the aerosol generating component of FIG. 6 with an outer wrap removed, according to one embodiment of the present disclosure; and

FIG. 8 is a bar graph representation of the aerosol formation properties of an aerosol generating component according to one or more embodiments of the present disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to percent is intended to mean percent by weight unless otherwise indicated.

As described hereinafter, example embodiments of the present disclosure relate to an aerosol generating component comprising a substrate which is substantially free of solid tobacco material, and which carries at least one aerosol forming material. In one embodiment, the substrate comprises about 60% dry weight or more of one or more fillers, based on the total dry weight of the substrate, wherein calcium carbonate comprises at least about 80% by dry weight of the one or more fillers; and at least one binder. In another embodiment, the substrate comprises about 60% dry weight or more of one or more fillers, based on the total dry weight of the substrate; and at least one binder, the at least one binder comprising a combination of hydroxypropyl cellulose (HPC) and hydroxypropyl methylcellulose (HPMC).

Further example embodiments of the present disclosure relate to an aerosol delivery device comprising the aerosol generating component as disclosed herein; a heat source configured to heat the aerosol forming materials carried in the substrate portion to form an aerosol; and an aerosol pathway extending from the aerosol generating component to a mouth-end of the aerosol delivery device.

### Aerosol Generating Components and Aerosol Delivery Devices

Some embodiments of aerosol generating components according to the present disclosure use electrical energy to heat a material to form an inhalable substance (e.g., electrically heated tobacco products). Other embodiments of aerosol generating components according to the present disclosure use an ignitable heat source to heat a material (generally without combusting the material to any significant degree) to form an inhalable substance (e.g., carbon heated tobacco products). The material is typically heated without combusting the material to any significant degree. Components of such systems have the form of articles that are sufficiently compact to be considered hand-held devices. That is, use of components of certain example aerosol delivery devices does not result in the production of smoke in the sense that aerosol results principally from by-products of combustion or pyrolysis of tobacco, but rather, use of those systems results in the production of vapors resulting from volatilization or vaporization of certain components incorporated therein. In some example embodiments, components of aerosol delivery devices may be characterized as electronic cigarettes, and those electronic cigarettes typically incorporate tobacco and/or components derived from tobacco, and hence deliver tobacco derived components in aerosol form.

Aerosol generating components of certain example aerosol delivery devices may provide many of the sensations (e.g., inhalation and exhalation rituals, types of tastes or flavors, organoleptic effects, physical feel, use rituals, visual cues such as those provided by visible aerosol, and the like) of smoking a cigarette, cigar or pipe that is employed by lighting and burning tobacco (and hence inhaling tobacco smoke), without any substantial degree of combustion of any component thereof. For example, the user of an aerosol delivery device in accordance with some example embodiments of the present disclosure can hold and use that component much like a smoker employs a traditional type of smoking article, draw on one end of that piece for inhalation of aerosol produced by that piece, take or draw puffs at selected intervals of time, and the like.

While the systems are generally described herein in terms of embodiments associated with aerosol delivery devices and/or aerosol generating components such as so-called “e-cigarettes” or “tobacco heating products,” it should be understood that the mechanisms, components, features, and methods may be embodied in many different forms and associated with a variety of articles. For example, the description provided herein may be employed in conjunction with embodiments of traditional smoking articles (e.g., cigarettes, cigars, pipes, etc.), heat-not-burn cigarettes, and related packaging for any of the products disclosed herein. Accordingly, it should be understood that the description of the mechanisms, components, features, and methods disclosed herein are discussed in terms of embodiments relating to aerosol delivery devices by way of example only, and may be embodied and used in various other products and methods.

Aerosol delivery devices and/or aerosol generating components of the present disclosure may also be characterized as being vapor-producing articles or medicament delivery articles. Thus, such articles or devices may be adapted so as to provide one or more substances (e.g., flavors and/or pharmaceutical active ingredients) in an inhalable form or state. For example, inhalable substances may be substantially in the form of a vapor (i.e., a substance that is in the gas phase at a temperature lower than its critical point). Alternatively, inhalable substances may be in the form of an aerosol (i.e., a suspension of fine solid particles or liquid droplets in a gas). For purposes of simplicity, the term “aerosol” as used herein is meant to include vapors, gases and aerosols of a form or type suitable for human inhalation, whether or not visible, and whether or not of a form that might be considered to be smoke-like. The physical form of the inhalable substance is not necessarily limited by the nature of the inventive devices but rather may depend upon the nature of the medium and the inhalable substance itself as to whether it exists in a vapor state or an aerosol state. In some embodiments, the terms “vapor” and “aerosol” may be interchangeable. Thus, for simplicity, the terms “vapor” and “aerosol” as used to describe aspects of the disclosure are understood to be interchangeable unless stated otherwise.

In some embodiments, aerosol delivery devices of the present disclosure may comprise some combination of a power source (e.g., an electrical power source), at least one control component (e.g., means for actuating, controlling, regulating and ceasing power for heat generation, such as by controlling electrical current flow from the power source to other components of the article, e.g., a microprocessor, individually or as part of a microcontroller), a heat source (e.g., an electrical resistance heating element or other component and/or an inductive coil or other associated components and/or one or more radiant heating elements), and an aerosol generating component that includes a substrate portion capable of yielding an aerosol upon application of sufficient heat. Note that it is possible to physically combine one or more of the above-noted components. For instance, in certain embodiments, a conductive heater trace can be printed on the surface of a substrate material as described herein (e.g., a cellulosic film) using a conductive ink such that the heater trace can be powered by the power source and used as the resistance heating element. Example conductive inks include graphene inks and inks containing various metals, such as inks including silver, gold, palladium, platinum, and alloys or other combinations thereof (e.g., silver-palladium or silver-platinum inks), which can be printed on a surface using processes such as gravure printing, flexographic printing, off-set printing, screen printing, ink-jet printing, or other appropriate printing methods.

In various embodiments, a number of these components may be provided within an outer body or shell, which, in some embodiments, may be referred to as a housing. The overall design of the outer body or shell may vary, and the format or configuration of the outer body that may define the overall size and shape of the aerosol delivery device may vary. Although other configurations are possible, in some embodiments an elongated body resembling the shape of a cigarette or cigar may be a formed from a single, unitary housing or the elongated housing can be formed of two or more separable bodies. For example, an aerosol delivery device may comprise an elongated shell or body that may be substantially tubular in shape and, as such, resemble the shape of a conventional cigarette or cigar. In one example, all of the components of the aerosol delivery device are contained within one housing or body. In other embodiments, an aerosol delivery device may comprise two or more housings that are joined and are separable. For example, an aerosol delivery device may possess at one end a control body comprising a housing containing one or more reusable components (e.g., an accumulator such as a rechargeable battery and/or rechargeable supercapacitor, and various electronics for controlling the operation of that article), and at the other end and removably coupleable thereto, an outer body or shell containing a disposable portion (e.g., a disposable flavor-containing aerosol generating component).

In other embodiments, aerosol generating components of the present disclosure may generally include an ignitable heat source configured to heat a substrate material. The substrate material and/or at least a portion of the heat source may be covered in an outer wrap, or wrapping, a casing, a component, a module, a member, or the like. The overall design of the enclosure is variable, and the format or configuration of the enclosure that defines the overall size and shape of the aerosol generating component is also variable. Although other configurations are possible, it may be desirable, in some aspects, that the overall design, size, and/or shape of these embodiments resemble that of a conventional cigarette or cigar. In various aspects, the heat source may be capable of generating heat to aerosolize a substrate material that comprises, for example, a substrate material associated with an aerosol forming material, an extruded structure and/or substrate, tobacco and/or a tobacco related material, such as a material that is found naturally in tobacco that is isolated directly from the tobacco or synthetically prepared, in a solid or liquid form (e.g., beads, sheets, shreds, a wrap), or the like.

More specific formats, configurations and arrangements of various substrate materials, aerosol generating components, and components within aerosol delivery devices of the present disclosure will be evident in light of the further disclosure provided hereinafter. Additionally, the selection of various aerosol delivery device components may be appreciated upon consideration of the commercially available electronic aerosol delivery devices. Further, the arrangement of the components within the aerosol delivery device may also be appreciated upon consideration of the commercially available electronic aerosol delivery devices.

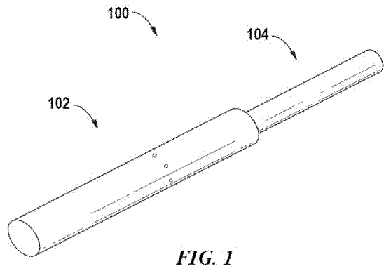

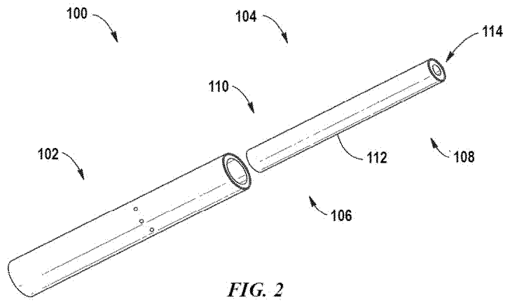

In this regard, FIG. 1 illustrates an aerosol delivery device 100 according to an example embodiment of the present disclosure. The aerosol delivery device 100 may include a control body 102 and an aerosol generating component 104. In various embodiments, the aerosol generating component 104 and the control body 102 may be permanently or detachably aligned in a functioning relationship. In this regard, FIG. 1 illustrates the aerosol delivery device 100 in a coupled configuration, whereas FIG. 2 illustrates the aerosol delivery device 100 in a decoupled configuration. Various mechanisms may connect the aerosol generating component 104 to the control body 102 to result in a threaded engagement, a press-fit engagement, an interference fit, a sliding fit, a magnetic engagement, or the like.

In various embodiments, the aerosol delivery device 100 according to than example embodiment of the present disclosure may have a variety of overall shapes, including, but not limited to an overall shape that may be defined as being substantially rod-like or substantially tubular shaped or substantially cylindrically shaped. In the embodiments of FIGS. 1-2, the device 100 has a substantially round cross-section; however, other cross-sectional shapes (e.g., oval, square, triangle, etc.) also are encompassed by the present disclosure. For example, in some embodiments one or both of the control body 102 or the aerosol generating component 104 (and/or any subcomponents) may have a substantially rectangular shape, such as a substantially rectangular cuboid shape (e.g., similar to a USB flash drive). In other embodiments, one or both of the control body 102 or the aerosol generating component 104 (and/or any subcomponents) may have other hand-held shapes. For example, in some embodiments the control body 102 may have a small box shape, various pod mod shapes, or a fob-shape. Thus, such language that is descriptive of the physical shape of the article may also be applied to the individual components thereof, including the control body 102 and the aerosol generating component 104.

Alignment of the components within the aerosol delivery device of the present disclosure may vary across various embodiments. In some embodiments, the substrate portion may be positioned proximate a heat source so as to maximize aerosol delivery to the user. Other configurations, however, are not excluded. Generally, the heat source may be positioned sufficiently near the substrate portion so that heat from the heat source can volatilize the substrate portion (as well as, in some embodiments, one or more flavorants, active ingredients, or the like that may likewise be provided for delivery to a user) and form an aerosol for delivery to the user. When the heat source heats the substrate portion, an aerosol is formed, released, or generated in a physical form suitable for inhalation by a consumer. It should be noted that the foregoing terms are meant to be interchangeable such that reference to release, releasing, releases, or released includes form or generate, forming or generating, forms or generates, and formed or generated. Specifically, an inhalable substance is released in the form of a vapor or aerosol or mixture thereof, wherein such terms are also interchangeably used herein except where otherwise specified.

As noted above, the aerosol delivery device 100 of various embodiments may incorporate a battery and/or other electrical power source to provide current flow sufficient to provide various functionalities to the aerosol delivery device, such as powering of the heat source, powering of control systems, powering of indicators, and the like. As will be discussed in more detail below, the power source may take on various embodiments. The power source may be able to deliver sufficient power to rapidly activate the heat source to provide for aerosol formation and power the aerosol delivery device through use for a desired duration of time. In some embodiments, the power source is sized to fit conveniently within the aerosol delivery device so that the aerosol delivery device can be easily handled. Examples of useful power sources include lithium-ion batteries that are typically rechargeable (e.g., a rechargeable lithium-manganese dioxide battery). In particular, lithium polymer batteries can be used as such batteries can provide increased safety. Other types of batteries—e.g., N50-AAA CADNICA nickel-cadmium cells—may also be used. Additionally, an example power source is of a sufficiently light weight to not detract from a desirable smoking experience. Some examples of possible power sources are described in U.S. Pat. No. 9,484,155 to Peckerar et al., and U.S. Pat. App. Pub. No. 2017/0112191 to Sur et al., the disclosures of which are incorporated herein by reference in their respective entireties.

In specific embodiments, one or both of the control body 102 and the aerosol generating component 104 may be referred to as being disposable or as being reusable. For example, the control body 102 may have a replaceable battery or a rechargeable battery, solid-state battery, thin-film solid-state battery, rechargeable supercapacitor or the like, and thus may be combined with any type of recharging technology, including connection to a wall charger, connection to a car charger (i.e., cigarette lighter receptacle), and connection to a computer, such as through a universal serial bus (USB) cable or connector (e.g., USB 2.0, 3.0, 3.1, USB Type-C), connection to a photovoltaic cell (sometimes referred to as a solar cell) or solar panel of solar cells, a wireless charger, such as a charger that uses inductive wireless charging (including for example, wireless charging according to the Qi wireless charging standard from the Wireless Power

Consortium (WPC)), or a wireless radio frequency (RF) based charger. An example of an inductive wireless charging system is described in U.S. Pat. App. Pub. No. 2017/0112196 to Sur et al., which is incorporated herein by reference in its entirety. Further, in some embodiments, the aerosol generating component 104 may comprise a single-use device. A single use component for use with a control body is disclosed in U.S. Pat. No. 8,910,639 to Chang et al., which is incorporated herein by reference in its entirety.

In further embodiments, the power source may also comprise a capacitor. Capacitors are capable of discharging more quickly than batteries and can be charged between puffs, allowing the battery to discharge into the capacitor at a lower rate than if it were used to power the heat source directly. For example, a supercapacitor—e.g., an electric double-layer capacitor (EDLC)—may be used separate from or in combination with a battery. When used alone, the supercapacitor may be recharged before each use of the article. Thus, the device may also include a charger component that can be attached to the smoking article between uses to replenish the supercapacitor.

Further components may be utilized in the aerosol delivery device of the present disclosure. For example, the aerosol delivery device may include a flow sensor that is sensitive either to pressure changes or air flow changes as the consumer draws on the article (e.g., a puff-actuated switch). Other possible current actuation/deactuation mechanisms may include a temperature actuated on/off switch or a lip pressure actuated switch. An example mechanism that can provide such puff-actuation capability includes a Model 163PC01D36 silicon sensor, manufactured by the MicroSwitch division of Honeywell, Inc., Freeport, Ill. Representative flow sensors, current regulating components, and other current controlling components including various microcontrollers, sensors, and switches for aerosol delivery devices are described in U.S. Pat. No. 4,735,217 to Gerth et al., U.S. Pat. Nos. 4,922,901, 4,947,874, and 4,947,875, all to Brooks et al., U.S. Pat. No. 5,372,148 to McCafferty et al., U.S. Pat. No. 6,040,560 to Fleischhauer et al., U.S. Pat. No. 7,040,314 to Nguyen et al., and U.S. Pat. No. 8,205,622 to Pan, all of which are incorporated herein by reference in their entireties. Reference is also made to the control schemes described in U.S. Pat. No. 9,423,152 to Ampolini et al., which is incorporated herein by reference in its entirety.

In another example, an aerosol delivery device may comprise a first conductive surface configured to contact a first body part of a user holding the device, and a second conductive surface, conductively isolated from the first conductive surface, configured to contact a second body part of the user. As such, when the aerosol delivery device detects a change in conductivity between the first conductive surface and the second conductive surface, a vaporizer is activated to vaporize a substance so that the vapors may be inhaled by the user holding unit. The first body part and the second body part may be a lip or parts of a hand(s). The two conductive surfaces may also be used to charge a battery contained in the personal vaporizer unit. The two conductive surfaces may also form, or be part of, a connector that may be used to output data stored in a memory. Reference is made to U.S. Pat. No. 9,861,773 to Terry et al., which is incorporated herein by reference in its entirety.

In addition, U.S. Pat. No. 5,154,192 to Sprinkel et al. discloses indicators for smoking articles; U.S. Pat. No. 5,261,424 to Sprinkel, Jr. discloses piezoelectric sensors that can be associated with the mouth-end of a device to detect user lip activity associated with taking a draw and then trigger heating of a heating device; U.S. Pat. No. 5,372,148 to McCafferty et al. discloses a puff sensor for controlling energy flow into a heating load array in response to pressure drop through a mouthpiece; U.S. Pat. No. 5,967,148 to Harris et al. discloses receptacles in a smoking device that include an identifier that detects a non-uniformity in infrared transmissivity of an inserted component and a controller that executes a detection routine as the component is inserted into the receptacle; U.S. Pat. No. 6,040,560 to Fleischhauer et al. describes a defined executable power cycle with multiple differential phases; U.S. Pat. No. 5,934,289 to Watkins et al. discloses photonic-optronic components; U.S. Pat. No. 5,954,979 to Counts et al. discloses means for altering draw resistance through a smoking device; U.S. Pat. No. 6,803,545 to Blake et al. discloses specific battery configurations for use in smoking devices; U.S. Pat. No. 7,293,565 to Griffen et al. discloses various charging systems for use with smoking devices; U.S. Pat. No. 8,402,976 to Fernando et al. discloses computer interfacing means for smoking devices to facilitate charging and allow computer control of the device; U.S. Pat. No. 8,689,804 to Fernando et al. discloses identification systems for smoking devices; and PCT Pat. App. Pub. No. WO 2010/003480 by Flick discloses a fluid flow sensing system indicative of a puff in an aerosol generating system; all of the foregoing disclosures being incorporated herein by reference in their entireties.

Further examples of components related to electronic aerosol delivery articles and disclosing materials or components that may be used in the present device include U.S. Pat. No. 4,735,217 to Gerth et al.; U.S. Pat. No. 5,249,586 to Morgan et al.; U.S. Pat. No. 5,666,977 to Higgins et al.; U.S. Pat. No. 6,053,176 to Adams et al.; U.S. Pat. No. 6,164,287 to White; U.S. Pat No. 6,196,218 to Voges; U.S. Pat. No. 6,810,883 to Felter et al.; U.S. Pat. No. 6,854,461 to Nichols; U.S. Pat. No. 7,832,410 to Hon; U.S. Pat. No. 7,513,253 to Kobayashi; U.S. Pat. No.

7,896,006 to Hamano; U.S. Pat. No. 6,772,756 to Shayan; U.S. Pat. Nos. 8,156,944 and 8,375,957 to Hon; U.S. Pat. No. 8,794,231 to Thorens et al.; U.S. Pat. No. 8,851,083 to Oglesby et al.; U.S. Pat. Nos. 8,915,254 and 8,925,555 to Monsees et al.; U.S. Pat. No. 9,220,302 to DePiano et al.; U.S. Pat. App. Pub. Nos. 2006/0196518 and 2009/0188490 to Hon; U.S. Pat. App. Pub. No. 2010/0024834 to Oglesby et al.; U.S. Pat. App. Pub. No. 2010/0307518 to Wang; PCT Pat. App. Pub. No. WO 2010/091593 to Hon; and PCT Pat. App. Pub. No. WO 2013/089551 to Foo, each of which is incorporated herein by reference in its entirety. Further, U.S. Pat. App. Pub. No. 2017/0099877 to Worm et al. discloses capsules that may be included in aerosol delivery devices and fob-shape configurations for aerosol delivery devices, and is incorporated herein by reference in its entirety. A variety of the materials disclosed by the foregoing documents may be incorporated into the present devices in various embodiments, and all of the foregoing disclosures are incorporated herein by reference in their entireties.

Referring to FIG. 2, in the depicted embodiment, the aerosol generating component 104 comprises a heated end 106, which is configured to be inserted into the control body 102, and a mouth end 108, upon which a user draws to create the aerosol. At least a portion of the heated end 106 includes a substrate portion 110. In some embodiments, the substrate portion 110 comprises a substrate carrying the aerosol forming material, each as further described herein below. In various embodiments, the aerosol generating component 104, or a portion thereof, may be wrapped in an exterior overwrap material 112. In various embodiments, the mouth end 108 of the aerosol generating component 104 may include a filter 114, which may, for example, be made of a cellulose acetate or polypropylene material. The filter 114 may additionally or alternatively contain strands of tobacco containing material, such as described in U.S. Pat. No. 5,025,814 to Raker et al., which is incorporated herein by reference in its entirety. In various embodiments, the filter 114 may increase the structural integrity of the mouth end of the aerosol generating component 104, and/or provide filtering capacity, if desired, and/or provide resistance to draw. In some embodiments, the filter may comprise discrete segments. For example, some embodiments may include a segment providing filtering, a segment providing draw resistance, a hollow segment providing a space for the aerosol to cool, a segment providing increased structural integrity, other filter segments, and any one or any combination of the above.

In some embodiments, the material of the exterior overwrap 112 may comprise a material that resists transfer of heat, which may include a paper or other fibrous material, such as a cellulose material. The exterior overwrap material may also include at least one filler material imbedded or dispersed within the fibrous material. In various embodiments, the filler material may have the form of water insoluble particles. Additionally, the filler material may incorporate inorganic components. In various embodiments, the exterior overwrap may be formed of multiple layers, such as an underlying, bulk layer and an overlying layer, such as a typical wrapping paper in a cigarette. Such materials may include, for example, lightweight “rag fibers” such as flax, hemp, sisal, rice straw, and/or esparto. The exterior overwrap may also include a material typically used in a filter element of a conventional cigarette, such as cellulose acetate. Further, an excess length of the exterior overwrap at the mouth end 108 of the aerosol generating component may function to simply separate the substrate portion 110 from the mouth of a consumer or to provide space for positioning of a filter material, as described below, or to affect draw on the article or to affect flow characteristics of the vapor or aerosol leaving the device during draw. Further discussions relating to the configurations for exterior overwrap materials that may be used with the present disclosure may be found in U.S. Pat. No. 9,078,473 to Worm et al., which is incorporated herein by reference in its entirety.

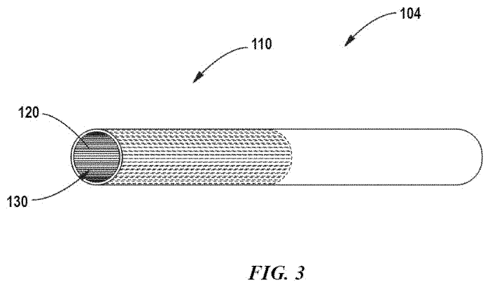

FIG. 3 illustrates a perspective schematic view of an aerosol generating component according to an example embodiment of the disclosure. In particular, FIG. 3 illustrates the aerosol generating component 104 having a substrate portion 110 that comprises a series of overlapping layers 130 of a substrate in sheet form 120. With reference to the description above, in the depicted embodiment, the substrate sheet 120 comprises a film or layer as disclosed herein. In various embodiments, the term “overlapping layers” may also include bunched, crumpled, crimped, and/or otherwise gathered layers in which the individual layers may not be obvious.

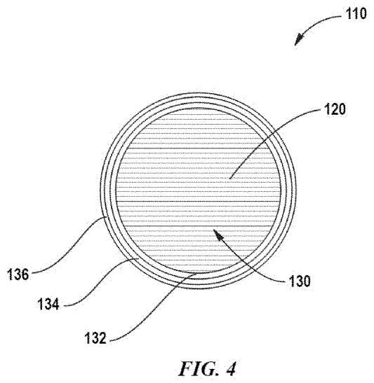

For example, FIG. 4 illustrates a schematic cross-section drawing of a substrate portion 110 of an aerosol generating component 104 according to an example embodiment of the present disclosure. In particular, FIG. 4 illustrates the substrate portion 110, which comprises a series of overlapping layers 130 of the substrate sheet 120. In the depicted embodiment, at least a portion of the overlapping layers 130 is substantially surrounded about its outer surface with a first cover layer 132. Although in various embodiments the composition of the first cover layer 132 may vary, in the depicted embodiment the first cover layer 132 comprises a combination of a fibrous material, the aerosol forming materials, and a binder material. Reference is made to the discussions herein relating possible aerosol forming materials and binder materials.

In various embodiments, the first cover layer 132 may be constructed via a casting process, such as that described in U.S. Pat. No. 5,697,385 to Seymour et al., the disclosure of which is incorporated herein by reference in its entirety.

In the depicted embodiment, at least a portion of the overlapping layers 130 and the first cover layer 132 are substantially surrounded about an outer surface with a second cover layer 134. Although the composition of the second cover layer 134 may vary, in the depicted embodiment the second cover layer 134 comprises a metal foil material, such as an aluminum foil material. In other embodiments, the second cover layer may comprise other materials, including, but not limited to, a copper material, a tin material, a gold material, an alloy material, a ceramic material, or other thermally conductive amorphous carbon-based material, and/or any combinations thereof. The depicted embodiment further includes a third cover layer 136, which substantially surrounds the overlapping layers 130, first cover layer 132, and the second cover layer 134, about an outer surface thereof. In the depicted embodiment, the third cover layer 136 comprises a paper material, such as a conventional cigarette wrapping paper. In various embodiments, the paper material may comprise rag fibers, such as non-wood plant fibers, and may include flax, hemp, sisal, rice straw, and/or esparto fibers.

In various embodiments, other components may exist between the substrate portion 110 and the mouth end 108 of the aerosol generating component 104. For example, in some embodiments one or any combination of the following may be positioned between the substrate portion 110 and the mouth end 108 of the aerosol generating component 104: an air gap; a hollow tube structure; phase change materials for cooling air; flavor releasing media; ion exchange fibers capable of selective chemical adsorption; aerogel particles as filter medium; and other suitable materials. Some examples of possible phase change materials include, but are not limited to, salts, such as AgNO₃, AlCl₃, TaCl₃, InCl₃, SnCl₂, AlI₃, and TiI₄; metals and metal alloys such as selenium, tin, indium, tin-zinc, indium-zinc, or indium-bismuth; and organic compounds such as D-mannitol, succinic acid, p-nitrobenzoic acid, hydroquinone and adipic acid. Other examples are described in U.S. Pat. No. 8,430,106 to Potter et al., which is incorporated herein by reference in its entirety.

As will be discussed in more detail below, the presently disclosed aerosol generating component is configured for use with a conductive and/or inductive heat source to heat a substrate material to form an aerosol. In various embodiments, a conductive heat source may comprise a heating assembly that comprises a resistive heating member. Resistive heating members may be configured to produce heat when an electrical current is directed therethrough. Electrically conductive materials useful as resistive heating members may be those having low mass, low density, and moderate resistivity and that are thermally stable at the temperatures experienced during use. Useful heating members heat and cool rapidly, and thus provide for the efficient use of energy. Rapid heating of the member may be beneficial to provide almost immediate volatilization of an aerosol forming materials in proximity thereto. Rapid cooling prevents substantial volatilization (and hence waste) of the aerosol forming materials during periods when aerosol formation is not desired. Such heating members may also permit relatively precise control of the temperature range experienced by the aerosol forming materials, especially when time based current control is employed. Useful electrically conductive materials are typically chemically non-reactive with the materials being heated (e.g., aerosol forming materials and other inhalable substance materials) so as not to adversely affect the flavor or content of the aerosol or vapor that is produced. Some example, non-limiting, materials that may be used as the electrically conductive material include carbon, graphite, carbon/graphite composites, metals, ceramics such as metallic and non-metallic carbides, nitrides, oxides, silicides, inter-metallic compounds, cermets, metal alloys, and metal foils. In particular, refractory materials may be useful. Various, different materials can be mixed to achieve the desired properties of resistivity, mass, and thermal conductivity. In specific embodiments, metals that can be utilized include, for example, nickel, chromium, alloys of nickel and chromium (e.g., nichrome), and steel. Materials that can be useful for providing resistive heating are described in U.S. Pat. No. 5,060,671 to Counts et al.; U.S. Pat. Nos. 5,093,894 to Deevi et al.; 5,224,498 to Deevi et al.; 5,228,460 to Sprinkel Jr., et al.; 5,322,075 to Deevi et al.; U.S. Pat. No. 5,353,813 to Deevi et al.; U.S. Pat. No. 5,468,936 to Deevi et al.; U.S. Pat. No. 5,498,850 to Das; U.S. Pat. No. 5,659,656 to Das; U.S. Pat. No. 5,498,855 to Deevi et al.; U.S. Pat. No. 5,530,225 to Hajaligol; U.S. Pat. No. 5,665,262 to Hajaligol; U.S. Pat. No. 5,573,692 to Das et al.; and U.S. Pat. No. 5,591,368 to Fleischhauer et al., the disclosures of which are incorporated herein by reference in their entireties.

In various embodiments, a heating member may be provided in a variety of forms, such as in the form of a foil, a foam, a mesh, a hollow ball, a half ball, discs, spirals, fibers, wires, films, yarns, strips, ribbons, or cylinders. Such heating members often comprise a metal material and are configured to produce heat as a result of the electrical resistance associated with passing an electrical current therethrough. Such resistive heating members may be positioned in proximity to, and/or in direct contact with, the substrate portion. For example, in one embodiment, a heating member may comprise a cylinder or other heating device located in the control body 102, wherein the cylinder is constructed of one or more conductive materials, including, but not limited to, copper, aluminum, platinum, gold, silver, iron, steel, brass, bronze, carbon (e.g., graphite), or any combination thereof In various embodiments, the heating member may also be coated with any of these or other conductive materials. The heating member may be located proximate an engagement end of the control body 102, and may be configured to substantially surround a portion of the heated end 106 of the aerosol generating component 104 that includes the substrate portion 110. In such a manner, the heating member may be located proximate the substrate portion 110 of the aerosol generating component 104 when the aerosol generating component 104 is inserted into the control body 102. In other examples, at least a portion of a heating member may penetrate at least a portion of an aerosol generating component (such as, for example, one or more prongs and/or spikes that penetrate an aerosol generating component), when the aerosol generating component is inserted into the control body. Although in some embodiments the heating member may comprise a cylinder, it should be noted that in other embodiments, the heating member may take a variety of forms and, in some embodiments, may make direct contact with and/or penetrate the substrate portion.

As described above, in addition to being configured for use with a conductive heat source, the presently disclosed aerosol generating component may also be configured for use with an inductive heat source to heat a substrate portion to form an aerosol. In various embodiments, an inductive heat source may comprise a resonant transformer, which may comprise a resonant transmitter and a resonant receiver (e.g., a susceptor). In some embodiments, the resonant transmitter and the resonant receiver may be located in the control body 102. In other embodiments, the resonant receiver, or a portion thereof, may be located in the aerosol generating component 104. For example, in some embodiments, the control body 102 may include a resonant transmitter, which, for example, may comprise a foil material, a coil, a cylinder, or other structure configured to generate an oscillating magnetic field, and a resonant receiver, which may comprise one or more prongs that extend into the substrate portion or are surrounded by the substrate portion. In some embodiments, the aerosol generating component is in intimate contact with the resonant receiver.

In other embodiments, a resonant transmitter may comprise a helical coil configured to circumscribe a cavity into which an aerosol generating component, and in particular, a substrate portion of an aerosol generating component, is received. In some embodiments, the helical coil may be located between an outer wall of the device and the receiving cavity. In one embodiment, the coil winds may have a circular cross section shape; however, in other embodiments, the coil winds may have a variety of other cross section shapes, including, but not limited to, oval shaped, rectangular shaped, L-shaped, T-shaped, triangular shaped, and combinations thereof. In another embodiment, a pin may extend into a portion of the receiving cavity, wherein the pin may comprise the resonant transmitter, such as by including a coil structure around or within the pin. In various embodiments, an aerosol generating component may be received in the receiving cavity wherein one or more components of the aerosol generating component may serve as the resonant receiver. In some embodiments, the aerosol generating component comprises the resonant receiver. Other possible resonant transformer components, including resonant transmitters and resonant receivers, are described in U.S. Pat. App. Pub. No. 2019/0124979 to Sebastian et al., which is incorporated herein by reference in its entirety.

In one aspect of the disclosure is provided an aerosol generating component comprising a substrate carrying at least one aerosol forming material (forming substrate portion 110). As used herein, the term “carrying” refers to any manner of associating the at least one aerosol forming material with the substrate. For example, carrying encompasses the at least one aerosol forming material impregnated, imbibed, disposed, supported, or otherwise included in or on the substrate. The substrate and aerosol forming materials are further described herein below.

### Substrate

The substrate may comprise a variety of materials, alone or in combinations. The substrate is typically substantially free of solid tobacco material. The substrate of the disclosure is characterized, for example, as completely free or substantially free of solid tobacco material (other than purified nicotine as an active ingredient). For example, substrates of the present disclosure may be characterized as having less than 1% by dry weight, or less than 0.5% by dry weight, or less than 0.1% by dry weight of solid tobacco material, or 0% by dry weight of solid tobacco material, based on the total dry weight of the substrate. By “solid tobacco material” is meant tobacco materials such as tobacco pulp, fiber, marumerized tobacco, and the like. Reference to “substantially free of solid tobacco material” does not exclude tobacco extracts, either in liquid form or as dried powder tobacco extract.

The substrate of the disclosure generally comprises one or more fillers and at least one binder. Each of the one or more fillers and at least one binder is described further herein below, along with additional substrate components.

### Fillers

Substrates as disclosed herein comprise one or more fillers. The at least one filler may comprise materials such as starches, sugars, sugar alcohols, wood fibers, inorganic substances, inert materials, and the like.

The amount of filler can vary. In some embodiments, the substrate comprises about 60% dry weight or more of one or more fillers, based on the total dry weight of the substrate. For example, in some embodiments, the substrate comprises at least about 60%, at least about 65%, at least about 70%, at least about 75%, at least about 80%, at least about 85%, or at least about 90% dry weight of the one or more fillers, based on the total dry weight of the substrate. In some embodiments, the substrate comprises about 60%, about 65%, about 70%, about 75%, about 80%, about 85%, or about 90% dry weight of the one or more fillers, based on the total dry weight of the substrate. In some embodiments, the substrate comprises from about 60 to about 80% dry weight of the one or more fillers, based on the total dry weight of the substrate

In some embodiments, the at least one filler comprises a starch, including native and modified starches. “Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the beads based on the ability of the starch material to impart a specific organoleptic property to the beads. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Suitable starches include, but are not limited to, corn starch, rice starch, and modified food starches. Certain starches are modified starches. A modified starch has undergone one or more structural modifications, often designed to alter its high heat properties. Some starches have been developed by genetic modifications, and are considered to be “modified” starches. Other starches are obtained and subsequently modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, enzyme treatment, acetylation, hydroxypropylation, and/or partial hydrolysis. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

In some embodiments, the one or more fillers comprises corn starch, rice starch or rice flour, modified food starch, or a combination thereof. In some embodiments, the one or more fillers is rice starch or rice flour. In other embodiments, the substrate is substantially or completely free of rice starch and rice flour. By “substantially free” of rice starch and rice flour is meant that no rice starch or flour has been intentionally added, beyond trace amounts that may be naturally present in e.g., another starch material. For example, certain embodiments may be characterized as having less than 0.1% by dry weight, or less than 0.01% by dry weight, or less than 0.001% by dry weight, or 0% by dry weight of rice starch and rice flour, based on the total dry weight of the substrate.

In some embodiments, the one or more fillers comprises a sugar. Suitable sugars include, but are not limited to, glucose, dextrose, fructose, maltose, and lactose.

In some embodiments, the one or more fillers comprises a sugar alcohol. Suitable sugar alcohols include, but are not limited to, sorbitol, mannitol, isomalt, maltitol, erythritol, and xylitol.

In some embodiments, the one or more fillers comprises a cellulose material, such as microcrystalline cellulose (“mcc”). The mcc may be synthetic or semi-synthetic, or it may be obtained entirely from natural celluloses. The mcc may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof In one embodiment, the substrate comprises mcc as the filler.

In some embodiments, the one or more fillers comprises wood fibers. For example, in some embodiments, the one or more fillers comprises, on a dry weight basis, from about 0 to about 5% of wood fibers or wood-derived fibers, for example, about 0%, about 1%, about 2%, about 3%, about 4%, or about 5% wood fibers or wood-derived fibers. In other embodiments, the substrate is substantially or completely free of wood fibers or wood pulp. By “substantially free” of wood fibers or pulp is meant that no wood fibers or pulp have been intentionally added, beyond trace amounts that may be naturally present in e.g., a botanical or other plant material. For example, certain embodiments may be characterized as having less than 0.1% by dry weight, or less than 0.01% by dry weight, or less than 0.001% by dry weight, or 0% by dry weight of wood fibers or pulp, based on the total dry weight of the substrate.

In some embodiments, the one or more fillers comprise an inorganic substance or inert substance, such as, but not limited to, chitosan, carbons (graphite, diamond, fullerenes, graphene), quartz, granite, diatomaceous earth, calcium carbonate, calcium phosphate, clays, crustacean and other marine shells, or combinations thereof.

In some embodiments, the one or more fillers comprise maltodextrin, dextrose, calcium phosphate, lactose, sugar alcohols, microcrystalline cellulose, or a combination thereof.

In other embodiments, the one or more fillers comprise calcium carbonate. In some embodiments, the substrate comprises about 60% dry weight or more of one or more fillers, based on the total dry weight of the substrate, wherein calcium carbonate comprises at least about 80% by dry weight of the one or more fillers. In some embodiments, the substrate comprises at least about 55% by dry weight of calcium carbonate, based on the total dry weight of the substrate. In some embodiments, the substrate comprises at least about 60% by dry weight of calcium carbonate, based on the total dry weight of the substrate. In some embodiments, the substrate comprises from about 55%, about 56%, about 57%, about 58%, or about 59%, to about 60%, about 61%, about 62%, or about 63% by dry weight calcium carbonate, based on the total dry weight of the substrate.

In some embodiments, in addition to calcium carbonate, the one or more fillers further comprise maltodextrin, dextrose, calcium phosphate, lactose, sugar alcohols, microcrystalline cellulose, wood fibers, and combinations thereof.

### Binders

The substrate as disclosed herein comprises at least one binder. A binder (or combination of binders) may be employed in certain embodiments, in amounts sufficient to provide the desired physical attributes and physical integrity to the substrate. The amount of binder utilized can vary, but is typically up to about 30 dry weight percent, and certain embodiments are characterized by a binder content of at least about 0.1% by dry weight, such as about 1 to about 30% by dry weight, or about 1 to about 10% by dry weight, or about 1 to about 5% by dry weight, based on the total dry weight of the substrate.

Typical binders can be organic or inorganic, or a combination thereof. Representative binders include povidone, sodium alginate, pectin, gums, carrageenan, pullulan, zein, cellulose derivatives, and the like, and combinations thereof In some implementations, combinations or blends of two or more binder materials may be employed. Other examples of binder materials are described, for example, in U.S. Pat. No. 5,101,839 to Jakob et al.; and U.S. Pat. No. 4,924,887 to Raker et al., each of which is incorporated herein by reference in its entirety.

In some embodiments, the at least one binder is selected from the group consisting of alginates, starches, gums, pullulan, zein, carrageenan, cellulose derivatives, povidone, and combinations thereof In some embodiments, the at least one binder is selected from the group consisting of alginate salts, cellulose ethers, and combinations thereof.

In some embodiments, the at least one binder is an alginate, such as ammonium alginate, propylene glycol alginate, potassium alginate, or sodium alginate. Alginates, and particularly high viscosity alginates, may be employed in conjunction with controlled levels of free calcium ions. In some embodiments, the substrate comprises, on a dry weight basis, from about 1 to about 35% of an alginate, for example, from about 1 to about 20% by dry weight, or from about 4 to about 10% by dry weight of alginate, based on the total dry weight of the substrate.

In some embodiments, the at least one binder is a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. In some embodiments, binder comprises xanthan gum, guar gum, gum Arabic, locust bean gum, gum tragacanth, or a combination thereof.

In some embodiments, the at least one binder is a carrageenan. In some embodiments, the at least one binder is agar.

In some embodiments, the at least one binder is one or more cellulose derivatives (e.g., a single cellulose derivative or a combination of several cellulose derivatives, such as two or three, for example). The quantity of cellulose derivative present in the substrate may vary. In some embodiments, the substrate comprises, on a dry weight basis, from about 0 to about 5% of the one or more cellulose derivatives, for example, about 0%, about 1%, about 2%, about 3%, about 4%, or about 5% of the one or more cellulose derivatives, based on the total dry weight of the substrate. It is to be understood that in embodiments where the substrate comprises more than one cellulose derivative, the stated weight basis of the one or more cellulose derivatives of from about 0% to about 5% reflects the total dry weight of the combination of cellulose derivatives, based on the total dry weight of the substrate.

In some embodiments, the cellulose derivative is a cellulose ether, meaning a cellulose polymer with the hydrogen of one or more hydroxyl groups in the cellulose structure replaced with an alkyl, hydroxyalkyl, or aryl group. In some embodiments, the cellulose derivative is a hydroxyalkyl cellulose ether. Non-limiting examples of such cellulose derivatives include methylcellulose, hydroxypropylcellulose (“HPC”), hydroxypropylmethylcellulose (“HPMC”), and hydroxyethyl cellulose. Suitable cellulose ethers include hydroxypropylcellulose, such as Klucel H from Aqualon Co.; hydroxypropylmethylcellulose, such as Methocel K4MS from DuPont; hydroxyethylcellulose, such as Natrosol 250 MRCS from Aqualon Co.; methylcellulose, such as Methocel A4M, K4M, and E15 from DuPont.; and sodium carboxymethylcellulose, such as CMC 7HF, CMC 7LF, and CMC 7H4F from Aqualon Co. In some embodiments, the at least one binder is a cellulose ether selected from the group consisting of methylcellulose, hydroxypropylcellulose, hydroxypropylmethylcellulose, hydroxyethyl cellulose, and combinations thereof. In some embodiments, the at least one binder comprises a combination of HPC and HPMC. In some embodiments, the at least one binder is a combination of HPC and HPMC. Surprisingly, it has been found that in some embodiments, a combination of HPC and HPMC is particularly useful in providing desirable properties to substrates of the disclosure, such as maintaining the desired shape and consistency of, for example, extruded substrates having a center hole. In some embodiments, a weight ratio of HPC to HPMC is at least about 1:1, for example, from about 1:1 to about 5:1, or from about 2:1 to about 4:1, or about 3:1.

In some embodiments, the substrate may be characterized as substantially or completely free of carboxymethylcellulose (CMC). By “substantially free” of CMC is meant that no CMC has been intentionally added, beyond trace amounts that may be naturally present in e.g., another cellulose ether. For example, certain embodiments may be characterized as having less than 0.1% by dry weight, or less than 0.01% by dry weight, or less than 0.001% by dry weight, or 0% by dry weight of CMC, based on the total dry weight of the substrate.

### Tobacco-Derived Materials

In some embodiments, the substrate comprises a tobacco extract, such as an aqueous tobacco extract, added either as a component of the aerosol forming material, or added separately (e.g., during substrate preparation, or impregnated in the substrate after formation). “Tobacco extract” as used herein refers to the isolated components of a tobacco material that are extracted from solid tobacco pulp by a solvent (e.g., water) that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in US Pat. Nos. 4,144,895 to Fiore; 4,150,677 to Osborne, Jr. et al.; 4,267,847 to Reid; 4,289,147 to Wildman et al.; 4,351,346 to Brummer et al.; 4,359,059 to Brummer et al.; 4,506,682 to Muller; 4,589,428 to Keritsis; 4,605,016 to Soga et al.; 4,716,911 to Poulose et al.; 4,727,889 to Niven, Jr. et al.; 4,887,618 to Bernasek et al.; 4,941,484 to Clapp et al.; 4,967,771 to Fagg et al.; 4,986,286 to Roberts et al.; 5,005,593 to Fagg et al.; 5,018,540 to Grubbs et al.; 5,060,669 to White et al.; 5,065,775 to Fagg; 5,074,319 to White et al.; 5,099,862 to White et al.; 5,121,757 to White et al.; 5,131,414 to Fagg; 5,131,415 to Munoz et al.; 5,148,819 to Fagg; 5,197,494 to Kramer; 5,230,354 to Smith et al.; 5,234,008 to Fagg; 5,243,999 to Smith; 5,301,694 to Raymond et al.; 5,318,050 to Gonzalez-Parra et al.; 5,343,879 to Teague; 5,360,022 to Newton; 5,435,325 to Clapp et al.; 5,445,169 to Brinkley et al.; 6,131,584 to Lauterbach; 6,298,859 to Kierulff et al.; 6,772,767 to Mua et al.; and 7,337,782 to Thompson, all of which are incorporated by reference herein.

In some embodiments, the substrate comprises a tobacco extract, in aqueous or dry powder form, in an amount of from about 1 to about 5% by dry weight, based on the total dry weight of the substrate. In other embodiments, the substrate is substantially or completely free of any tobacco-derived materials, including extracts. By “substantially free” of tobacco-derived materials is meant that no tobacco-derived material has been intentionally added, beyond trace amounts that may be naturally present in e.g., another botanical or plant-derived material. For example, certain embodiments may be characterized as having less than 0.1% by dry weight, or less than 0.01% by dry weight, or less than 0.001% by dry weight, or 0% by dry weight of tobacco-derived materials, based on the total dry weight of the substrate.

### Non-Tobacco Botanicals

In some embodiments, the substrate comprises a non-tobacco botanical, added either as a component of the aerosol forming material, or added separately (e.g., during substrate preparation, or impregnated in the substrate after formation). As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, or other treatment processes capable of altering the chemical nature of the material). For the purposes of the present disclosure, a “botanical material” includes but is not limited to “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). The botanical materials used in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.”

Non-limiting examples of botanical materials include without limitation acai berry, alfalfa, allspice, annatto seed, apricot oil, basil, bee balm, wild bergamot, black pepper, blueberries, borage seed oil, bugleweed, cacao, calamus root, cannabis/hemp, catnip, catuaba, cayenne pepper, chaga mushroom, chervil, cinnamon, dark chocolate, coffee, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum.

The quantity of botanical material present may vary, and when present, is generally less than about 30%. or less than about 20% by dry weight of the substrate, based on the total dry weight of the substrate. For example, a botanical material may be present in a quantity of from about 0.1%, about 0.5%, about 1%, or about 5%, to about 10%, about 20%, or about 30% by dry weight of the substrate, based on the total dry weight of the substrate.

### Active Ingredient

In certain embodiments, the substrate may comprise one or more active ingredients, added either as a component of the aerosol forming material, or added separately (e.g., during substrate preparation, or impregnated in the substrate after formation). As used herein, an “active ingredient” refers to one or more substances belonging to any of the following categories: API (active pharmaceutical substances), food additives, herbal materials, natural medicaments, and naturally occurring substances that can have an effect on humans. The active ingredient can be any known agent adapted for therapeutic, prophylactic, or diagnostic use. These can include, for example, synthetic organic compounds, proteins and peptides, polysaccharides and other sugars, lipids, inorganic compounds, and nucleic acid sequences, having therapeutic, prophylactic, or diagnostic activity. Active ingredients include, but are not limited to, a nicotine component, botanical ingredients (e.g., lavender, peppermint, chamomile, basil, rosemary, ginger, cannabis, ginseng, maca, and tisanes), stimulants (e.g., caffeine and guarana), amino acids (e.g., taurine, theanine, phenylalanine, tyrosine, and tryptophan) and/or pharmaceutical, nutraceutical, and medicinal ingredients (e.g., vitamins, such as B6, B12, and C, and/or cannabinoids, such as tetrahydrocannabinol (THC) and cannabidiol (CBD)). The particular percentages and choice of ingredients will vary depending upon the desired flavor, texture, and other characteristics. Example active ingredients would include any ingredient known to impact one or more biological functions within the body, such as ingredients that furnish pharmacological activity or other direct effect in the diagnosis, cure, mitigation, treatment, or prevention of disease, or which affect the structure or any function of the body of humans or other animals (e.g., provide a stimulating action on the central nervous system, have an energizing effect, an antipyretic or analgesic action, or an otherwise useful effect on the body).

The quantity of active ingredient present may vary, and when present, is generally less than about 30%, or less than about 20% by total weight of the substrate carrying the aerosol forming material. For example, an active ingredient may be present in a quantity of from about 0.1%, about 0.5%, about 1%, or about 5%, to about 10%, about 20%, or about 30% by total weight of the substrate carrying the aerosol forming material.

In some embodiments, the active ingredient comprises one or more herbal materials. For the purposes of the present disclosure, the term “herbal materials” refers to seed-producing plants that do not develop persistent woody tissue and are often valued for their medicinal or sensory characteristics (e.g., teas or tisanes). Certain herbal materials, as the plant material or an extract thereof, have found use in traditional herbal medicine. Non-limiting examples of herbal materials or herbal-derived materials include hemp, eucalyptus, rooibos, fennel, citrus, cloves, lavender, peppermint, chamomile, basil, rosemary, ginger, turmeric, green tea, white mulberry, cannabis, cocoa, ashwagandha, baobab, chlorophyll, cordyceps, damiana, ginseng, guarana, and maca.

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis.

When present, a stimulant or combination of stimulants (e.g., caffeine, theacrine, and combinations thereof) is typically at a concentration of from about 0.1% w/w to about 15% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the substrate.

In certain embodiments, the active ingredient comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HIVIR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the substrate carrying the aerosol forming material, such as in a range from about 0.001% to about 10%, based on the total weight of the substrate carrying the aerosol forming material. In some embodiments, the nicotine component is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the substrate carrying the aerosol forming material. In some embodiments, the nicotine component is present in a concentration from about 0.1% w/w to about 3% by weight, such as, e.g., from about from about 0.1% w/w to about 2.5%, from about 0.1% to about 2.0%, from about 0.1% to about 1.5%, or from about 0.1% to about 1% by weight, calculated as the free base and based on the total weight of the substrate carrying the aerosol forming material.

In some embodiments, the substrate of the disclosure can be characterized as completely free or substantially free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical or herbal material. For example, certain embodiments can be characterized as having less than 0.001% by weight of nicotine, or less than 0.0001%, or even 0% by weight of nicotine, calculated as the free base, and based on the total weight of the substrate carrying the aerosol forming material.

In some embodiments, the active ingredient comprises one or more cannabinoids. In some embodiments, the cannabinoid comprises cannabidiol (CBD), tetrahydrocannabinol (THC), or a combination thereof.

### Flavorant

In some embodiments, the substrate comprises a flavorant, added either as a component of the aerosol forming material, or added separately (e.g., during substrate preparation, or impregnated in the substrate after formation). As used herein, reference to a “flavorant” refers to compounds or components that can be aerosolized and delivered to a user and which impart a sensory experience in terms of taste and/or aroma. Some examples of flavorants include, but are not limited to, vanillin, ethyl vanillin, cream, tea, coffee, fruit (e.g., apple, cherry, strawberry, peach and citrus flavors, including lime and lemon), maple, menthol, mint, peppermint, spearmint, wintergreen, nutmeg, clove, lavender, cardamom, ginger, honey, anise, sage, rosemary, hibiscus, rose hip, yerba mate, guayusa, honeybush, rooibos, yerba santa, bacopa monniera, gingko biloba, withania somnifera, cinnamon, sandalwood, jasmine, cascarilla, cocoa, licorice, and flavorings and flavor packages of the type and character traditionally used for the flavoring of cigarette, cigar, and pipe tobaccos. Syrups, such as high fructose corn syrup, also can be employed. Some examples of plant-derived compositions that may be suitable are disclosed in U.S. Pat. No. 9,107,453 and U.S. Pat. App. Pub. No. 2012/0152265 both to Dube et al., the disclosures of which are incorporated herein by reference in their entireties. The selection of such further components is variable based upon factors such as the sensory characteristics that are desired for the smoking article, their affinity for the substrate material, their solubility, and other physiochemical properties. The present disclosure is intended to encompass any such further components that are readily apparent to those skilled in the art of tobacco and tobacco-related or tobacco-derived products. See, e.g., Gutcho, Tobacco Flavoring Substances and Methods, Noyes Data Corp. (1972) and Leffingwell et al., Tobacco Flavoring for Smoking Products (1972), the disclosures of which are incorporated herein by reference in their entireties. It should be noted that reference to a flavorant should not be limited to any single flavorant as described above, and may, in fact, represent a combination of one or more flavorants. Additional flavorants, flavoring agents, additives, and other possible enhancing constituents are described in U.S. Pat. App. Pub. No. 2019/0082735 to Phillips et al., which is incorporated herein by reference in its entirety.

The quantity of flavorant present may vary, and when present, is generally less than about 30%, or less than about 20% by weight of the substrate, based on the total weight of the substrate carrying the aerosol forming material. For example, a flavorant may be present in a quantity of from about 0.1%, about 0.5%, about 1%, or about 5%, to about 10%, about 20%, or about 30% by weight of the substrate, based on the total weight of the substrate carrying the aerosol forming material.

### Other Components

In some embodiments, the substrate may further comprise a burn retardant material, conductive fibers or particles for heat conduction/induction, or any combination thereof. One example of a burn retardant material is ammonium phosphate. In some embodiments, other flame/burn retardant materials and additives may be included within the substrate, and may include organo-phosphorus compounds, borax, hydrated alumina, graphite, potassium, silica, tripolyphosphate, dipentaerythritol, pentaerythritol, and polyols. Other burn retardant materials, such as nitrogenous phosphonic acid salts, mono-ammonium phosphate, ammonium polyphosphate, ammonium bromide, ammonium borate, ethanolammonium borate, ammonium sulphamate, halogenated organic compounds, thiourea, and antimony oxides may also be used. In each aspect of flame-retardant, burn-retardant, and/or scorch-retardant materials used in the substrate material and/or other components (whether alone or in combination with each other and/or other materials), the desirable properties are independent of and resistant to undesirable off-gassing or melting-type behavior. Various manners and methods for incorporating tobacco into smoking articles, and particularly smoking articles that are designed so as to not purposefully burn virtually all of the tobacco within those smoking articles are set forth in U.S. Pat. No. 4,947,874 to Brooks et al.; U.S. Pat. No. 7,647,932 to Cantrell et al.; U.S. Pat. No. 8,079,371 to Robinson et al.; U.S. Pat. No. 7,290,549 to Banerjee et al.; and U.S. Pat. App. Pub. No. 2007/0215167 to Crooks et al.; the disclosures of which are incorporated herein by reference in their entireties.

The substrate may also include conductive fibers or particles for heat conduction or heating by induction. In some embodiments, the conductive fibers or particles may be arranged in a substantially linear and parallel pattern. In some embodiments, the conductive fibers or particles may have a substantially random arrangement. In some embodiments, the conductive fibers or particles may be constructed of or more of an aluminum material, a stainless steel material, a copper material, a carbon material, and a graphite material. In some embodiments, one or more conductive fibers or particles with different Curie temperatures may be included in the substrate material to facilitate heating by induction at varying temperatures.

In still other implementations, the substrate material may comprise inorganic fibers of various types (e.g., fiber glass, metal wires/screens, etc.) and/or (organic) synthetic polymers. In various implementations, these “fibrous” materials could be unstructured (e.g., randomly distributed) or structured (e.g., a wire mesh).

### Aerosol Forming Material

Aerosol generating components as disclosed herein comprise a substrate carrying one or more aerosol forming materials. The amount of aerosol forming material that is carried by the substrate is such that the aerosol generating component provides acceptable sensory and desirable performance characteristics. For example, in certain embodiments, sufficient amounts of aerosol forming material are employed in order to provide for the generation of a visible mainstream aerosol that in many regards resembles the appearance of tobacco smoke. The amount of aerosol forming materials carried by the aerosol generating component (e.g., the substrate carrying the aerosol forming material) may be dependent upon factors such as the number of puffs desired per aerosol generating component.

In some embodiments, the substrate carries the aerosol forming material at a loading of at least about 10% by weight, of at least about 15% by weight, at least about 20% by weight, at least about 25% by weight, at least about 30% by weight, at least about 35% by weight, at least about 40% by weight, at least about 45% by weight, at least about 50% by weight, at least about 55% by weight, or at least about 60% by weight, based on a total weight of the substrate carrying the aerosol forming material. Example ranges of total aerosol forming materials include about 15% to about 60% by weight, such as about 15% to about 55%, or about 15% to about 25%, based on the total weight of the substrate carrying the aerosol forming material. Methods for loading (e.g., impregnating) aerosol forming materials into or onto substrate portions are described in U.S. Pat. No. 9,974,334 to Dooly et al., and U.S. Pub. Pat. App. Nos. 2015/0313283 to Collett et al. and 2018/0279673 to Sebastian et al., the disclosures of which are incorporated by reference herein in their entirety.

In any of the previous embodiments, the entire quantity of aerosol forming materials may be added prior to casting, extrusion, or the like, to form the aerosol generating component as disclosed herein. Alternatively, or in addition, a portion or all of the aerosol forming materials may be added to the substrate post-formation (e.g., one or more aerosol forming materials may be sprayed or otherwise disposed in or on the substrate material to form the aerosol generating component as disclosed herein.

The aerosol forming material may include water, polyhydric alcohols, polysorbates, sorbitan esters, fatty acids, fatty acid esters, waxes, terpenes, sugar alcohols, active ingredients, tobacco extract, or a combination of any thereof. Each of polyhydric alcohols, polysorbates, sorbitan esters, fatty acids, fatty acid esters, waxes, terpenes, sugar alcohols, and active ingredients are further described herein below.

### Water

In some embodiments, the aerosol forming material comprises water. In some embodiments, the aerosol forming material comprises water and further comprises polyhydric alcohols, polysorbates, sorbitan esters, fatty acids, fatty acid esters, waxes, terpenes, sugar alcohols, active ingredients, tobacco extract, or a combination of any thereof.

### Polyhydric Alcohols

In some embodiments, the aerosol forming material comprises one or more polyhydric alcohols. Examples of polyhydric alcohols include glycerol, propylene glycol, and other glycols such as 1,3-propanediol, diethylene glycol, and triethylene glycol. In some embodiments, the polyhydric alcohol is selected from the group consisting of glycerol, propylene glycol, 1,3-propanediol, diethylene glycol, triethylene glycol, and combinations thereof.

In some embodiments, the polyhydric alcohol is a mixture of glycerol and propylene glycol. The glycerol and propylene glycol may be present in various ratios, with either component predominating depending on the intended application. In some embodiments, the glycerol and propylene glycol are present in a ratio by weight of from about 3:1 to about 1:3. In some embodiments, the glycerol and propylene glycol are present in a ratio by weight of about 3:1, about 2:1, about 1:1, about 1:2, or about 1:3. In some embodiments, the glycerol and propylene glycol are present in a ratio of about 1:1 by weight.

### Polysorbates and Sorbitan Esters

In some embodiments, the aerosol forming material comprises one or more polysorbates. Examples of polysorbates include Polysorbate 60 (polyoxyethylene (20) sorbitan monostearate, Tween 60) and Polysorbate 80 (polyoxyethylene (20) sorbitan monooleate, Tween 80). The type of polysorbate used or the combination of polysorbates used depends on the intended effect desired, as the different polysorbates offer different attributes due to molecular sizes. For example, the polysorbate molecules increase in size from polysorbate 20 to polysorbate 80. Using smaller size polysorbate molecules creates less vapor quantity, but permits deeper lung penetration. This may be desirable when the user is in public where he would not want to create a large plume of “smoke” (i.e. vapors). Conversely, if a dense vapor is desired, which can convey the aromatic constituents of tobacco, larger polysorbate molecules can be employed. An additional benefit of using the polysorbate family of compounds is that the polysorbates lower the heat of vaporization of mixtures in which they are present.

In some embodiments, the aerosol forming material comprises one or more sorbitan esters. Examples of sorbitan esters include sorbitan monolaurate, sorbitan monostearate (Span 60), sorbitan monooleate (Span 20), and sorbitan tristearate (Span 65).

### Fatty Acids, Esters, and Waxes

In some embodiments, the aerosol forming material comprises one or more fatty acids. Fatty acids may include short-chain, long-chain, saturated, unsaturated, straight chain, or branched chain carboxylic acids. Fatty acids generally include C₄ to C₂₈ aliphatic carboxylic acids. Non-limiting examples of short- or long-chain fatty acids include butyric, propionic, valeric, oleic, linoleic, stearic, myristic, and palmitic acids.

In some embodiments, the aerosol forming material comprises one or more fatty acid esters. Examples of fatty acid esters include alkyl esters, monoglycerides, diglycerides, and triglycerides. Examples of monoglycerides include monolaurin and glycerol monostearate. Examples of triglycerides include triolein, tripalmitin, tristearate, glycerol tributyrate, and glycerol trihexanoate).

In some embodiments, the aerosol forming material comprises one or more waxes. Examples of waxes include carnauba, beeswax, candellila, which are known known to stabilize aerosol particles, improve palatability, or reduce throat irritation.

### Terpenes

In some embodiments, the aerosol forming material comprises one or more terpenes. As used herein, the term “terpenes” refers to hydrocarbon compounds produced by plants biosynthetically from isopentenyl pyrophosphate. Non-limiting examples of terpenes include limonene, pinene, farnesene, myrcene, geraniol, fennel, and cembrene.

### Sugar Alcohols

In some embodiments, the aerosol forming material comprises one or more sugar alcohols. Examples of sugar alcohols include sorbitol, erythritol, mannitol, maltitol, isomalt, and xylitol. Sugar alcohols may also serve as flavor enhancers to certain flavor compounds, e.g. menthol and other volatiles, and generally improve on mouthfeel, tactile sensation, throat impact, and other sensory properties, of the resulting aerosol.

### Form of Substrate

The form of the substrate may vary. In some embodiments, the substrate is in particulate form, shredded form, film form, paper process sheet form, cast sheet form, bead form, granular rod form, or extrudate form. In various embodiments, the form of the substrate may include gels, shreds, films, suspensions, extrusions, shavings, capsules, and/or particles (including pellets, beads, strips, or any desired particle shape of varying sizes) and combinations thereof.

### Method of Producing an Aerosol Generating Component

In another aspect is provided a method of producing an aerosol generating component comprising a substrate as disclosed herein. The method generally comprises making a substrate comprising about 60% dry weight or more of one or more fillers and at least one binder. The aerosol forming material may be added during formation of the substrate according to any of the disclosed preparative methods, may be added after formation of the substrate, or a combination thereof, as further described herein below. The substrates of the present disclosure, depending on the desired form, may be prepared by different methods and processes, such as cast sheet and granular extrusion, each of which is further described herein below.

### Cast Sheet

In some embodiments, the substrate is prepared using cast sheet technology to make the substrate in the form of a flat sheet. The preparative method generally comprises producing a substrate mixture about 60% dry weight or more of one or more fillers, based on the total dry weight of the substrate, wherein calcium carbonate comprises at least about 80% by dry weight of the one or more fillers; and at least one binder; casting the substrate mixture on to a support surface; solidifying the substrate mixture to form the substrate; and reducing the substrate into a plurality of reduced portions configured for insertion into an aerosol generating device. For example, in some embodiments the substrate components as disclosed herein, optionally including the aerosol forming material, may be blended together to form a slurry, which may be cast onto a surface (such as, for example, a moving belt). The cast slurry may then experience one or more drying and/or doctoring steps such that the result is a relatively consistent thickness cast sheet. Other examples of casting and paper-making techniques are set forth in U.S. Pat. No. 4,674,519 to Keritsis et al.; U.S. Pat. No. 4,941,484 to Clapp et al.; U.S. Pat. No. 4,987,906 to Young et al.; U.S. Pat. No. 4,972,854 to Kiernan et al.; U.S. Pat. No. 5,099,864 to Young et al.; U.S. Pat. No. 5,143,097 to Sohn et al.; U.S. Pat. No. 5,159,942 to Brinkley et al.; U.S. Pat. No. 5,322,076 to Brinkley et al.; U.S. Pat. No. 5,339,838 to Young et al.; U.S. Pat. No. 5,377,698 to Litzinger et al.; U.S. Pat. No. 5,501,237 to Young; and U.S. Pat. No. 6,216,706 to Kumar; the disclosures of which is incorporated herein by reference in their entireties.

In some embodiments, the substrate is in flat sheet form. In some embodiments, the flat sheet is layered, for example, in a series of overlapping layers 130 of the flat sheet 120 as illustrated in FIG. 3. In some embodiments, the flat sheet may be bunched, crumpled, crimped, and/or otherwise gathered layers. In some embodiments, the flat sheet may further be reduced into cut rag or strips for inserting into the substrate-containing segment of an aerosol delivery device. The flat sheet may also be gathered or rolled into rod for insertion into the substrate-containing segment of an aerosol delivery device.

In some cast sheet embodiments, the binder comprises an alginate. Accordingly, in specific embodiments, the method comprises: hydrating an alginate (e.g., sodium or ammonium alginate) in water in a first mixing tank to form a hydrated alginate solution; mixing calcium carbonate in glycerol in a second mixing tank to create a calcium carbonate-glycerol slurry; transferring the hydrated alginate solution to the second mixing tank holding the calcium carbonate-glycerol slurry; mixing the hydrated alginate solution and the calcium carbonate slurry to form a final slurry; casting the substrate mixture on to a support surface; solidifying the substrate mixture to form the substrate; and optionally, reducing the substrate into a plurality of reduced portions configured for insertion into the aerosol generating device.

The mixing times and speeds can vary to suit a particular application. The various mixing steps can be at moderate and/or variable speeds (e.g., from about 50-500 RPM, such as from about 75-150 RPM) for the calcium carbonate-glycerol slurry, and can last from about 5 minutes to 60 minutes, such as about 15 minutes to 45 minutes or about 30 minutes. In some embodiments, the glycerol is slowly added to the calcium carbonate and gently mixed (e.g., at a speed of from about 50-150 RPM) for about 30 minutes. In some embodiments, glycerol and propylene glycol are premixed and slowly added to calcium carbonate and gently mixed (e.g., at a speed of from about 50-150 RPM) for about 30 minutes.

In some embodiments, hydrating the sodium alginate solution, is performed at with mixing at moderate to high speeds (e.g., from about 300 to about 3500 RPM, such as from 1000-2500 RPM) for a time of from about 10 minutes to about 60 minutes, such as from about 15 minutes to about 45 minutes. In some cases, e.g., hydrating the alginate and/or mixing the alginate-calcium-glycerol mixture, the mixing is performed under vacuum. In some embodiments, the alginate is slowly added to the water and hydrated in a high shear mixing tank for about 30-45 minutes under vacuum.

In some embodiments, the method further comprises mixing a wood pulp into the hydrated alginate solution. In some embodiments, the wood pulp is a pre-refined wood pulp (<0% Canadian Freeness measurement).

In some embodiments, the method further comprises mixing a rice starch or a flour (e.g. native or modified such as pre-gelatinized) to the calcium carbonate.

In some embodiments, the method may further comprise adding a tobacco extract powder and a menthol flavor in to the calcium carbonate-glycerol slurry, adding nicotine into the calcium carbonate-glycerol slurry, or both.

In some embodiments, casting the substrate mixture comprises casting the final slurry of the substrate mixture onto the support surface using a casting knife set at about a 1 to 3 mm gap opening.

In some embodiments, solidifying the substrate solution comprises drying the cast substrate mixture by heating.

In some embodiments, reducing the substrate comprises cutting the substrate into strips.

In some embodiments, the method further comprises removing the substrate from the support surface; optionally, drying the formed substrate; winding the substrate on a bobbin; and sealing the substrate within a container.

### Extrusion

In some embodiments, the substrate is prepared using extrusion technology. The preparative method generally comprises producing a substrate mixture comprising about 60% dry weight or more of one or more fillers, based on the total dry weight of the substrate, wherein calcium carbonate comprises at least about 80% by dry weight of the one or more fillers; and at least one binder; and forming a substrate from the substrate mixture, optionally including the aerosol forming material, via at least one of extrusion, marumerization, spheronization, or combinations thereof. The granular extrusion formulation may be similar to that of the cast sheet formulation, except, for example, that an alternate or additional binder (e.g., a cellulose derivative, such as carboxymethyl cellulose, hydroxy propylmethylcellulose, hydroxypropylcellulose, or combinations thereof) may be used therein.

In some embodiments, producing the substrate mixture comprises mixing a calcium carbonate in a glycerol in a plough mixer to create a calcium carbonate-glycerol slurry; mixing a carboxymethyl cellulose with water in a vessel to create a carboxymethyl cellulose slurry; adding the carboxymethyl cellulose slurry to the calcium carbonate-glycerol slurry; and mixing the carboxymethyl cellulose slurry and the calcium carbonate-glycerol slurry for a predetermined amount of time to form a final substrate mixture. In some embodiments, the predetermined amount of time ranges from about 10 to 60 minutes.

In some embodiments, the method further comprises mixing a rice starch or a flour (e.g. native or modified such as pre-gelatinized) to the calcium carbonate.

In other embodiments, producing the substrate mixture comprises mixing hydroxy propylmethylcellulose and/or hydroxypropylcellulose with glycerin to form a cellulose-glycerin slurry; adding the cellulose-glycerin slurry to calcium carbonate in a plough mixer; and mixing the cellulose-glycerin slurry and the calcium carbonate for a predetermined amount of time to form a final substrate mixture. In some embodiments, the predetermined amount of time ranges from about 10 to 60 minutes. In various embodiments, the mixing times (e.g., for mixing the calcium carbonate-glycerol slurry) can be about 10 minutes to 60 minutes, such as about 15 minutes to 45 minutes or about 20 minutes to 30 minutes. The mixing speeds can vary from about 50 RPM to 500 RPM, such as about 75 RPM to 150 RPM, or about 100 RPM. For mixing the carboxymethyl cellulose, hydroxypropylmethyl cellulose, and/or hydroxypropylcellulose—glycerol slurry, the mixing times can range from 10 minutes to 60 minutes, such as about 15 minutes to 45 minutes or about 30 minutes to 45 minutes. The mixing speeds can vary from about 400 RPM to 3500 RPM, such as about 1000 RPM to 2500 RPM, or about 1500-2500 RPM.

In some embodiments, the method further comprises mixing a rice starch or a rice flour (e.g. native or modified such as pre-gelatinized) to the calcium carbonate.

In some embodiments, the method further comprises adding a tobacco extract powder and a menthol flavor in to the calcium carbonate-glycerol slurry, adding nicotine into the calcium carbonate-glycerol slurry, or both.

In some embodiments, the substrate is prepared according to the described method, but in the absence of carboxymethylcellulose (CMC).

In some embodiments, the substrate mixture may be subject to spheronization or marumerization to produce round or ovoid shaped beads, or hair-like rods. Accordingly, in some embodiments, the method further comprises portioning the substrate mixture; transferring a portion of the substrate mixture to an extruder to form rods of the substrate mixture; transferring at least a portion of the extruded rods to a marumerizer to form rounded beads of the substrate mixture; and agglomerating the rods, the rounded beads, or a combination thereof.

In some embodiments, the substrate may be prepared by extrusion, followed by cutting or sizing to provide multiple size and/or shaped substrate pieces. The extrusion formulation may be similar to that of the granular extrusion formulation, except, for example, that yet another binder or binder combination (e.g., a combination of cellulose derivatives, such as hydroxypropyl methylcellulose and hydroxypropylcellulose) may be used therein. In some embodiments, the at least one binder comprises a combination of HPC and HPMC, or is a combination of HPC and HPMC. Surprisingly, it has been found that the combination of HPC and HPMC is particularly useful in extrusion methods, maintaining the desired shape and consistency of, for example, extruded substrates having a center hole. In some embodiments, a weight ratio of HPC to HPMC is at least about 1:1, such as about 1:1 to about 5:1, about 2:1 to about 4:1, or about 3:1.

In some embodiments, the method further comprises transferring the substrate mixture to an extruder; introducing water into the extruder to facilitate kneading, mixing and plasticization of the substrate mixture; extruding the substrate mixture to form a shaped substrate; and cutting the shaped substrate to form multiple substrate pieces of various sizes and shapes. In some embodiments, the method further comprises agglomerating at least a portion of the multiple substrate pieces to form a unitary substrate.

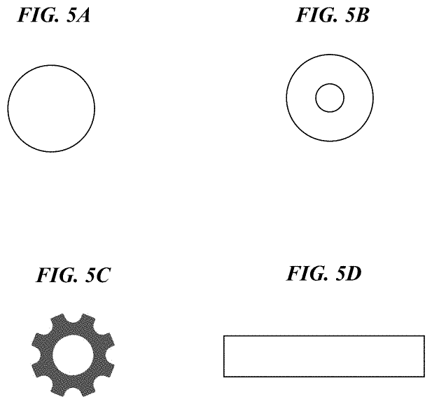

In some embodiments, the substrate can have an extruded form. In some embodiments, the substrate is formed into a substantially cylindrical shape. In some embodiments, the substrate may include a central orifice. In other embodiments, no central orifice is present. FIGS. 5A-5D illustrate perspective schematic views of non-limiting substrate extrudate embodiments. With reference to FIG. 5A, a non-limiting depiction of a substrate in extruded form of generally cylindrical shape is provided. In some embodiments, the extrudate has a grooved surface, a central orifice, or both. FIG. 5B depicts a non-limiting embodiment of a substrate in extruded form having a substantially cylindrical shape and having a central orifice. FIG. 5C depicts a non-limiting embodiment of a substrate in extruded form having a substantially cylindrical shape, a central orifice, and a grooved surface. A plurality of grooves extends longitudinally from a first end of the extruded substrate to an opposing second end of the extruded substrate. Although in the depicted embodiment, the grooves of the substrate are substantially equal in width and depth and are substantially equally distributed about a circumference of the substrate, other embodiments may include as few as two grooves, and still other embodiments may include as few as a single groove. Additional embodiments may include multiple grooves that may be of unequal width and/or depth, and which may be unequally spaced around a circumference of the substrate. In some embodiments, the substrate is in the form of a sheet, which may be extruded or cast. One non-limiting illustration of a sheet embodiment is provided in FIG. 5D, which shows a rectangular, flat embodiment.

### Substrate Loading

In various embodiments, the substrate may be associated with the aerosol forming material by impregnating the substrate with the aerosol forming material during preparation of the substrate material, after formation of the substrate material, or both. For example, in some embodiments, a portion of the aerosol forming material (e.g., glycerol or propylene glycol) is added to the slurry used to form the substrate during e.g., making of a sheet, and a second portion of the aerosol forming material (e.g., glycerol or propylene glycol) is added to the sheet as a top dressing (for example, by spraying) to form the substrate carrying the aerosol forming material. In other embodiments, the entirety of the aerosol forming material is added to the slurry used to form the substrate during the making of the substrate. In some embodiments, further aerosol forming materials may be impregnated in or on the substrate, either by adding further aerosol forming materials to the substrate forming slurry, or as a top dressing to the substrate. As one of skill will recognize, multiple permutations of methods for loading the substrate with the aerosol forming material is possible, depending on the specific substrate material, form, and the like. Accordingly, any such modifications are contemplated herein.

### Aerosol Delivery Device

As described herein, in another aspect is provided an aerosol delivery device comprising the aerosol generating component as disclosed herein; a heat source configured to heat the aerosol forming materials carried in the substrate portion to form an aerosol; and an aerosol pathway extending from the aerosol generating component to a mouth-end of the aerosol delivery device.

Although in some embodiments an aerosol generating component and a control body may be provided together as a complete smoking article or pharmaceutical delivery article generally, the components may be provided separately. For example, the present disclosure also encompasses a disposable unit for use with a reusable smoking article or a reusable pharmaceutical delivery article. In specific embodiments, such a disposable unit (which may be an aerosol generating component as illustrated in the appended figures) can comprise a substantially tubular shaped body having a heated end configured to engage the reusable smoking article or pharmaceutical delivery article, an opposing mouth end configured to allow passage of an inhalable substance to a consumer, and a wall with an outer surface and an inner surface that defines an interior space. Various embodiments of an aerosol generating component (or cartridge) are described in U.S. Pat. No. 9,078,473 to Worm et al., which is incorporated herein by reference in its entirety.

Although some figures described herein illustrate the control body and aerosol generating component in a working relationship, it is understood that the control body and the aerosol generating component may exist as individual devices. Accordingly, any discussion otherwise provided herein in relation to the components in combination also should be understood as applying to the control body and the aerosol generating component as individual and separate components.

In another aspect, the present disclosure may be directed to kits that provide a variety of components as described herein. For example, a kit may comprise a control body with one or more aerosol generating components. A kit may further comprise a control body with one or more charging components. A kit may further comprise a control body with one or more batteries. A kit may further comprise a control body with one or more aerosol generating components and one or more charging components and/or one or more batteries. In further embodiments, a kit may comprise a plurality of aerosol generating components. A kit may further comprise a plurality of aerosol generating components and one or more batteries and/or one or more charging components. In the above embodiments, the aerosol generating components or the control bodies may be provided with a heating member inclusive thereto. The inventive kits may further include a case (or other packaging, transporting, or storage component) that accommodates one or more of the further kit components. The case could be a reusable hard or soft container. Further, the case could be simply a box or other packaging structure.

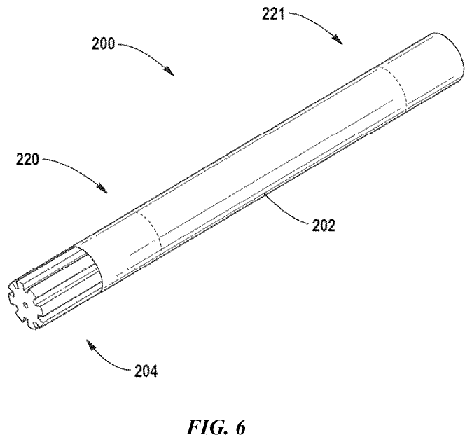

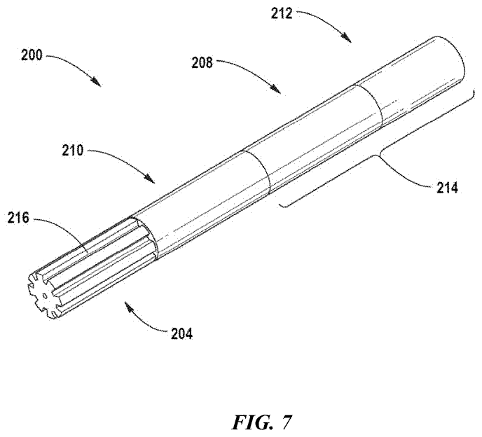

FIG. 6 illustrates a perspective view of an aerosol generating component, according to another example embodiment of the present disclosure, and FIG. 7 illustrates a perspective view of the aerosol generating component of FIG. 6 with an outer wrap removed. In particular, FIG. 6 illustrates an aerosol generating component 200 that includes an outer wrap 202, and FIG. 7 illustrates the aerosol generating component 200 wherein the outer wrap 202 is removed to reveal the other components of the aerosol generating component 200. In the depicted embodiment, the aerosol generating component 200 of the depicted embodiment includes a heat source 204, a substrate portion 210, an intermediate component 208, and a filter 212. In the depicted embodiment, the intermediate component 208 and the filter 212 together comprise a mouthpiece 214.

Although an aerosol deliver device and/or an aerosol generating component according to the present disclosure may take on a variety of embodiments, as discussed in detail below, the use of the aerosol delivery device and/or aerosol generating component by a consumer will be similar in scope. The foregoing description of use of the aerosol delivery device and/or aerosol generating component is applicable to the various embodiments described through minor modifications, which are apparent to the person of skill in the art in light of the further disclosure provided herein. The description of use, however, is not intended to limit the use of the articles of the present disclosure but is provided to comply with all necessary requirements of disclosure herein.

In various embodiments, the heat source 204 may be configured to generate heat upon ignition thereof In the depicted embodiment, the heat source 204 comprises a combustible fuel element that has a generally cylindrical shape and that incorporates a combustible carbonaceous material. In other embodiments, the heat source 204 may have a different shape, for example, a prism shape having a triangular, cubic or hexagonal cross-section. Carbonaceous materials generally have a high carbon content. Certain example carbonaceous materials may be composed predominately of carbon, and/or typically may have carbon contents of greater than about 60 percent, generally greater than about 70 percent, often greater than about 80 percent, and frequently greater than about 90 percent, on a dry weight basis.

In some instances, the heat source 204 may incorporate elements other than combustible carbonaceous materials (e.g., tobacco components, such as powdered tobaccos or tobacco extracts; flavoring agents; salts, such as sodium chloride, potassium chloride and sodium carbonate; heat stable graphite fibers; iron oxide powder; glass filaments; powdered calcium carbonate; alumina granules; ammonia sources, such as ammonia salts; binding agents, such as guar gum, ammonium alginate and sodium alginate; and/or phase change materials for lowering the temperature of the heat source, described herein above). Although specific dimensions of an applicable heat source may vary, in some embodiments, the heat source 204 may have a length in an inclusive range of approximately 7 mm to approximately 20 mm, and in some embodiments may be approximately 17 mm, and an overall diameter in an inclusive range of approximately 3 mm to approximately 8 mm, and in some embodiments may be approximately 4.8 mm (and in some embodiments, approximately 7 mm). Although in other embodiments, the heat source may be constructed in a variety of ways, in the depicted embodiment, the heat source 204 is extruded or compounded using a ground or powdered carbonaceous material, and has a density that is greater than about 0.5 g/cm³, often greater than about 0.7 g/cm³, and frequently greater than about 1 g/cm³, on a dry weight basis. See, for example, the types of fuel source components, formulations and designs set forth in U.S. Pat. No. 5,551,451 to Riggs et al. and U.S. Pat. No. 7,836,897 to Borschke et al., which are incorporated herein by reference in their entireties. Although in various embodiments, the heat source may have a variety of forms, including, for example, a substantially solid cylindrical shape or a hollow cylindrical (e.g., tube) shape, the heat source 204 of the depicted embodiment comprises an extruded monolithic carbonaceous material that has a generally cylindrical shape but with a plurality of grooves 216 extending longitudinally from a first end of the extruded monolithic carbonaceous material to an opposing second end of the extruded monolithic carbonaceous material. In some embodiments, the aerosol delivery device, and in particular, the heat source, may include a heat transfer component. In various embodiments, a heat transfer component may be proximate the heat source, and, in some embodiments, a heat transfer component may be located in or within the heat source. Some examples of heat transfer components are described in in U.S. Pat. App. Pub. No. 2019/0281891 to Hejazi et al., which is incorporated herein by reference in its entirety.

Although in the depicted embodiment, the grooves 216 of the heat source 204 are substantially equal in width and depth and are substantially equally distributed about a circumference of the heat source 204, other embodiments may include as few as two grooves, and still other embodiments may include as few as a single groove. Still other embodiments may include no grooves at all. Additional embodiments may include multiple grooves that may be of unequal width and/or depth, and which may be unequally spaced around a circumference of the heat source. In still other embodiments, the heat source may include flutes and/or slits extending longitudinally from a first end of the extruded monolithic carbonaceous material to an opposing second end thereof. In some embodiments, the heat source may comprise a foamed carbon monolith formed in a foam process of the type disclosed in U.S. Pat. No. 7,615,184 to Lobovsky, which is incorporated herein by reference in its entirety. As such, some embodiments may provide advantages with regard to reduced time taken to ignite the heat source. In some other embodiments, the heat source may be co-extruded with a layer of insulation (not shown), thereby reducing manufacturing time and expense. Other embodiments of fuel elements include carbon fibers of the type described in U.S. Pat. No. 4,922,901 to Brooks et al. or other heat source embodiments such as is disclosed in U.S. Pat. App. Pub. No. 2009/0044818 to Takeuchi et al., each of which is incorporated herein by reference in its entirety.

Generally, the heat source is positioned sufficiently near a substrate portion carrying one or more aerosol forming materials so that the aerosol formed/volatilized by the application of heat from the heat source to the aerosol forming materials (as well as any flavorants, medicaments, and/or the like that are likewise provided for delivery to a user) is deliverable to the user by way of the mouthpiece. That is, when the heat source heats the substrate portion, an aerosol is formed, released, or generated in a physical form suitable for inhalation by a consumer. It should be noted that the foregoing terms are meant to be interchangeable such that reference to release, releasing, releases, or released includes form or generate, forming or generating, forms or generates, and formed or generated. Specifically, an inhalable substance is released in the form of a vapor or aerosol or mixture thereof.

Referring back to FIGS. 6 and 7, the outer wrap 202 may be provided to engage or otherwise join together at least a portion of the heat source 204 with the substrate portion 210 and at least a portion of the mouthpiece 214. In various embodiments, the outer wrap 202 is configured to be retained in a wrapped position in any manner of ways including via an adhesive, or a fastener, and the like, to allow the outer wrap 202 to remain in the wrapped position. Otherwise, in some other aspects, the outer wrap 202 may be configured to be removable as desired. For example, upon retaining the outer wrap 202 in a wrapped position, the outer wrap 202 may be able to be removed from the heat source 204, the substrate portion 210, and/or the mouthpiece 214.

In some embodiments, in addition to the outer wrap 202, the aerosol delivery device may also include a liner that is configured to circumscribe the substrate portion 210 and at least a portion of the heat source 204. Although in other embodiments the liner may circumscribe only a portion of the length of the substrate portion 210, in some embodiments, the liner may circumscribe substantially the full length of the substrate portion 210. In some embodiments, the outer wrap material 202 may include the liner. As such, in some embodiments the outer wrap material 202 and the liner may be separate materials that are provided together (e.g., bonded, fused, or otherwise joined together as a laminate). In other embodiments, the outer wrap 202 and the liner may be the same material. In any event, the liner may be configured to thermally regulate conduction of the heat generated by the ignited heat source 204, radially outward of the liner. As such, in some embodiments, the liner may be constructed of a metal foil material, an alloy material, a ceramic material, or other thermally conductive amorphous carbon-based material, and/or an aluminum material, and in some embodiments may comprise a laminate. In some embodiments, depending on the material of the outer wrap 202 and/or the liner, a thin layer of insulation may be provided radially outward of the liner. Thus, the liner may advantageously provide, in some aspects, a manner of engaging two or more separate components of the aerosol generating component 200 (such as, for example, the heat source 204, the substrate portion 210, and/or a portion of the mouthpiece 214), while also providing a manner of facilitating heat transfer axially therealong, but restricting radially outward heat conduction.

As shown in FIG. 6, the outer wrap 202 (and, as necessary, the liner, and the substrate portion 210) may also include one or more openings formed therethrough that allow the entry of air upon a draw on the mouthpiece 214. In various embodiments, the size and number of these openings may vary based on particular design requirements. In the depicted embodiment, a plurality of openings 220 are located proximate an end of the substrate portion 210 closest to the heat source 204, and a plurality of separate cooling openings 221 are formed in the outer wrap 202 (and, in some embodiments, the liner) in an area proximate the filter 212 of the mouthpiece 214. Although other embodiments may differ, in the depicted embodiment, the openings 220 comprise a plurality of openings substantially evenly spaced about the outer surface of the aerosol generating component 200, and the openings 221 also comprise a plurality of openings substantially evenly spaced around the outer surface of the aerosol generating component 200. Although in various embodiments the plurality of openings may be formed through the outer wrap 202 (and, in some embodiments, the liner) in a variety of ways, in the depicted embodiment, the plurality of openings 220 and the plurality of separate cooling openings 221 are formed via laser perforation.

Referring back to FIG. 7, the aerosol generating component 200 of the depicted implementation also includes an intermediate component 208 and at least one filter 212. It should be noted that in various implementations, the intermediate component 208 or the filter 212, individually or together, may be considered a mouthpiece 214 of the aerosol generating component 200. Although in various implementations, neither the intermediate component nor the filter need be included, in the depicted implementation the intermediate component 208 comprises a substantially rigid member that is substantially inflexible along its longitudinal axis. In the depicted implementation, the intermediate component 208 comprises a hollow tube structure, and is included to add structural integrity to the aerosol generating component 200 and provide for cooling the produced aerosol. In some implementations, the intermediate component 208 may be used as a container for collecting the aerosol. In various implementations, such a component may be constructed from any of a variety of materials and may include one or more adhesives. Example materials include, but are not limited to, paper, paper layers, paperboard, plastic, cardboard, and/or composite materials. In the depicted implementation, the intermediate component 208 comprises a hollow cylindrical element constructed of a paper or plastic material (such as, for example, ethyl vinyl acetate (EVA), or other polymeric materials such as poly ethylene, polyester, silicone, etc. or ceramics (e.g., silicon carbide, alumina, etc.), or other acetate fibers), and the filter comprises a packed rod or cylindrical disc constructed of a gas permeable material (such as, for example, cellulose acetate or fibers such as paper or rayon, or polyester fibers).

As noted, in some implementations the mouthpiece 214 may comprise a filter 212 configured to receive the aerosol therethrough in response to the draw applied to the mouthpiece 214. In various implementations, the filter 212 is provided, in some aspects, as a circular disc radially and/or longitudinally disposed proximate the second end of the intermediate component 208. In this manner, upon draw on the mouthpiece 214, the filter 212 receives the aerosol flowing through the intermediate component 208 of the aerosol generating component 200. In some implementations, the filter 212 may comprise discrete segments. For example, some implementations may include a segment providing filtering, a segment providing draw resistance, a hollow segment providing a space for the aerosol to cool, a segment providing increased structural integrity, other filter segments, and any one or any combination of the above. In some implementations, the filter 212 may additionally or alternatively contain strands of tobacco containing material, such as described in U.S. Pat. No. 5,025,814 to Raker et al., which is incorporated herein by reference in its entirety.

In various implementations the size and shape of the intermediate component 208 and/or the filter 212 may vary, for example the length of the intermediate component 208 may be in an inclusive range of approximately 10 mm to approximately 30 mm, the diameter of the intermediate component 208 may be in an inclusive range of approximately 3 mm to approximately 8 mm, the length of the filter 212 may be in an inclusive range of approximately 10 mm to approximately 20 mm, and the diameter of the filter 212 may be in an inclusive range of approximately 3 mm to approximately 8 mm. In the depicted implementation, the intermediate component 208 has a length of approximately 20 mm and a diameter of approximately 4.8 mm (and in some implementations, approximately 7 mm), and the filter 212 has a length of approximately 15 mm and a diameter of approximately 4.8 mm (or in some implementations, approximately 7 mm).

In various implementations, ignition of the heat source 204 results in aerosolization of the aerosol forming materials associated with the substrate portion 210. In certain embodiments, the elements of the substrate portion 210 do not experience thermal decomposition (e.g., charring, scorching, or burning) to any significant degree, and the aerosolized components are entrained in the air that is drawn through the aerosol generating component 200, including the filter 212, and into the mouth of the user. In various implementations, the mouthpiece 214 (e.g., the intermediate component 208 and/or the filter 212) is configured to receive the generated aerosol therethrough in response to a draw applied to the mouthpiece 214 by a user. In some implementations, the mouthpiece 214 may be fixedly engaged to the substrate portion 210. For example, an adhesive, a bond, a weld, and the like may be suitable for fixedly engaging the mouthpiece 214 to the substrate portion 210. In one example, the mouthpiece 214 is ultrasonically welded and sealed to an end of the substrate portion 210.

Many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed herein and that modifications and other embodiments are intended to be included within the scope of the appended claims. Although specific terms are employed herein, they are used in a generic and descriptive sense only and not for purposes of limitation.

### EXAMPLES

Aspects of the present invention are more fully illustrated by the following examples, which are set forth to illustrate certain aspects of the present invention and are not to be construed as limiting thereof.

### Example 1

### Cast Sheet Substrate Embodiment

An example of a cast sheet substrate embodiment of the disclosure was prepared according to the formula provided in Table 1. Sodium alginate was slowly added to water and hydrated in a high shear mixing tank for 30 minutes under vacuum. In a separate mixing tank, calcium carbonate was slowly added to glycerol and then mixed gently for 30 minutes, forming a slurry. The hydrated alginate was then transferred into the calcium carbonate slurry tank, and both mixed for another 30 minutes under moderate mixing speeds and vacuum to obtain a final slurry. The final slurry was then cast onto a 22-inch-wide stainless steel conveyer belt using a casting knife set at 1-3 mm gap opening. The cast material or film was subsequently dried into a flat sheet by conveying the film through a 200 feet convection tunnel dryer, comprising multiple heated zones (e.g., ranging from 80-100° C.). The flat sheet was finally wound on a bobbin, and vacuum sealed in polyethylene bags to prevent moisture pickup and blocking during shipment.

The bobbins were subsequently unwound and the sheet cut into strips (e.g., about 25-20 cuts per square inch).

### Example 2

### Cast Sheet Substrate Embodiment

Another example of a cast sheet substrate embodiment of the disclosure was prepared according to the formula provided in Table 1. Example 2 was produced in a similar manner as to that outlined for Example 1, except that a tobacco extract powder and a menthol flavor were added to the calcium carbonate-glycerol slurry, which was then mixed for an additional 30 minutes.

### Example 3

### Cast Sheet Substrate Embodiment

Another example of a cast sheet substrate embodiment of the disclosure was prepared according to the formula provided in Table 1. Example 3 was produced in a similar manner as to that outlined for Example 2, except that ammonium alginate was substituted for the sodium alginate and nicotine for the tobacco extract. Example 3 also involved the introduction of a wood pulp (e.g., to increase flat sheet tensile strength), as well as the addition of propylene glycol to the glycerol. Pre-made wood pulp of zero freeness was added to the hydrated alginate and mixed for 30 minutes, before being added to the calcium carbonate-glycerol slurry.

TABLE 1
Formulations of cast sheet substrate embodiments

| | Example 1 | | Example 2 | | Example 3 | |
|---|---|---|---|---|---|---|
| | Percent | Batch | Percent | Batch | Percent | Batch |
| | composition | size | composition | size | composition | size |
| Component | (%) | (LB) | (%) | (LB) | (%) | (LB) |
| Calcium carbonate | 69.0 | 345.0 | 63.0 | 189.0 | 56.0 | 168.0 |
| Sodium alginate | 6.0 | 30.0 | 6.0 | 18.0 | | |
| Ammonium alginate | | | | | 6.0 | 18.0 |
| Wood pulp | | | | | 5.0 | |
| Glycerol | 25.0 | 125.0 | 25.0 | 75.0 | 20 | 60.0 |
| Propylene glycol | | | | | 10 | 30.0 |
| Tobacco extract | | | 5.0 | 15.0 | | |
| powder | | | | | | |
| Nicotine (USP) | | | | | 2.0 | 6.0 |
| Menthol | | | 1.0 | 3.0 | 1.0 | 3.0 |
| Total | 100.0 | 500.0 | 100.0 | 300.0 | 100.0 | 300.0 |
| Water | | 1500.0 | | 900.0 | | 1000.0 |

### Example 4

### Extruded Substrate (Granular Rods and Beads)

An example of an extruded substrate embodiment of the disclosure in the form of granular rods or beads was prepared according to the formula provided in Table 2. Calcium carbonate was weighed and transferred into a precision plough mixer (B&P Littleford. model FM130D) Glycerol was added and the mixture mixed at 100 rpm for 10 minutes. The mixer was stopped, and then a pre-made slurry of carboxymethyl cellulose was added and the contents mixed for another 20 minutes at 100 rpm. The pre-made slurry was prepared by mixing carboxymethyl cellulose with water in a vessel using a pitched fork propeller. This was carried out for 30 minutes. After mixing of the calcium carbonate-glycerol slurry and the carboxymethyl cellulose slurry for 20 minutes, the contents from the precision plough mixer were portioned and transferred into a multi grain extruder (Fuji Paudel Co. Ltd. model MG-55-1). The mass was extruded through a 2-3 mm doomed screen die, resulting in multi-grain (hair-like) shaped rods. The rods (or at least a portion thereof) were subsequently transferred into a laboratory marumerizer (Fuji Paudal Co. Ltd. model QJ-230T-2).

The rotating bowl of the marumerizer was used to reshape the rods into rounded beads. Subsequently, the beads were transferred into a fluidized bed agglomerator (Flo-Coater, Vector Corporation), and finally dried to 10% moisture with 60-70° C. heated air. Portions of the extruded rods not transferred to the marumerizer were also transferred and subsequently dried using the fluidized bed agglomerator.

### Example 5

### Extruded Substrate (Granular Rods and Beads)

Another example of an extruded substrate embodiment of the disclosure in the form of granular rods or beads was prepared according to the formula provided in Table 2. Example 5 was produced in a similar manner as to that outlined for Example 4, except that a portion of the calcium carbonate was replaced with rice starch/flour.

### Example 6

### Extruded Substrate (Granular Rods and Beads)

Another example of an extruded substrate embodiment of the disclosure in the form of granular rods or beads was prepared according to the formula provided in Table 2. Example 6 was produced in a similar manner to Example 5, except that a tobacco extract and a mint liquid flavoring were mixed with the glycerol.

TABLE 2
Formulation of beads and granular rod HNB substrates.

| | Example 4 | | Example 5 | | Example 6 | |
|---|---|---|---|---|---|---|
| | Percent | Batch | Percent | Batch | Percent | Batch |
| | composition | size | composition | size | composition | size |
| Component | (%) | (LB) | (%) | (LB) | (%) | (LB) |
| Calcium carbonate | 70.0 | 70.0 | 63.0 | 31.5 | 63.0 | 31.5 |
| Rice starch or rice | | | 15.0 | 7.5 | 10.0 | 5.0 |
| flour | | | | | | |
| Carboxymethyl | 5.0 | 5.0 | 2.0 | 1.0 | 2.0 | 1.0 |
| cellulose | | | | | | |
| Glycerol | 25.0 | 25.0 | 20.0 | 10.0 | 18.5 | 9.25 |
| Tobacco extract | | | | | 5.0 | 2.5 |
| powder | | | | | | |
| Mint flavor | | | | | 1.5 | 0.75 |
| Total | 100.0 | 100.0 | 100.0 | 50.0 | 100.0 | 50.0 |
| Water | | 35.0 | | 18.0 | | 18.0 |

### Example 7

### Extruded Substrate (Varied Shapes)

An example of an extruded substrate embodiment of the disclosure in various shaped forms was prepared according to the formula provided in Table 3. Hydroxypropylmethyl cellulose (HPMC) and hydroxypropylcellulose (HPC) were mixed with glycerin in a Hobart mixer for 20 minutes. The premix was then added to calcium carbonate in a model FM 130 D Littleford precision plough mixer and mixed at 100 rpm for 30 minutes. After 30 minutes, the plough mixer contents were transferred to a K-Tron hopper in-line with a twin screw model ZSK-25 Coperion extruder. The hopper contents were subsequently fed into the extruder comprised of eleven barrel sections (80-100° C.) and operated at a 75 rpm screw speed rate. Water was fed into the second barrel of the extrusion to facilitate kneading, mixing and plasticization of the dough.

Shaped dies (flat sheet, solid rods, rods with center holes or internal apertures and grooved edges, rods with grooved outer edges) were used to make various shaped extrudates. Except for the flat sheet, the other extrudates were cut upon exiting the die and immediately dried down to about 10-12% moisture using an infrared tunnel dryer (model Proj 0115, Glenroe Integrated Energy Delivery Systems).

### Example 8

### Extruded Substrate (Varied Shapes)

Another example of an extruded substrate embodiment of the disclosure in various shaped forms was prepared according to the formula provided in Table 3. Example 8 was produced in a similar manner as to that outlined in Example 7, except that a portion of the calcium carbonate was replaced with rice starch/flour.

### Example 9

### Extruded Substrate (Varied Shapes)

Another example of an extruded substrate embodiment of the disclosure in various shaped forms was prepared according to the formula provided in Table 3. Example 9 was produced in a similar manner as to that outlined in Example 8, except that a tobacco extract powder and a mint flavoring were mixed with the glycerin prior to the glycerin being added to HPMC and HPC in the Hobart mixer.

TABLE 3
Formulations for extruded HNB substrates

| | Example 7 | | Example 8 | | Example 9 | |
|---|---|---|---|---|---|---|
| | Percent | Batch | Percent | Batch | Percent | Batch |
| | composition | size | composition | size | composition | size |
| Component | (%) | (LB) | (%) | (LB) | (%) | (LB) |
| Calcium carbonate | 70.0 | 63.0 | 61.0 | 61.0 | 61.0 | 30.5 |
| Rice starch or rice | | | 15.0 | 15.0 | 10.0 | 5.0 |
| Flour | | | | | | |
| Hydroxypropyl | 2.5 | 2.5 | 2.0 | 2.0 | 2.0 | 1.0 |
| cellulose | | | | | | |
| Hydroxypropyl | 2.5 | 2.5 | 2.0 | 2.0 | 2.0 | 1.0 |
| methyl cellulose | | | | | | |
| Glycerin | 25.0 | 25.0 | 20.0 | 20.0 | 18.5 | 9.25 |
| Tobacco extract | | | | | 5.0 | 2.5 |
| powder | | | | | | |
| Mint flavor | | | | | 1.5 | 0.75 |
| Total | 100.0 | 100.0 | 100.0 | 100.0 | 100.0 | 50.0 |
| Water | | 35.0 | | 35.0 | | 15.0 |

### Example 10

### Aerosol Formation Properties

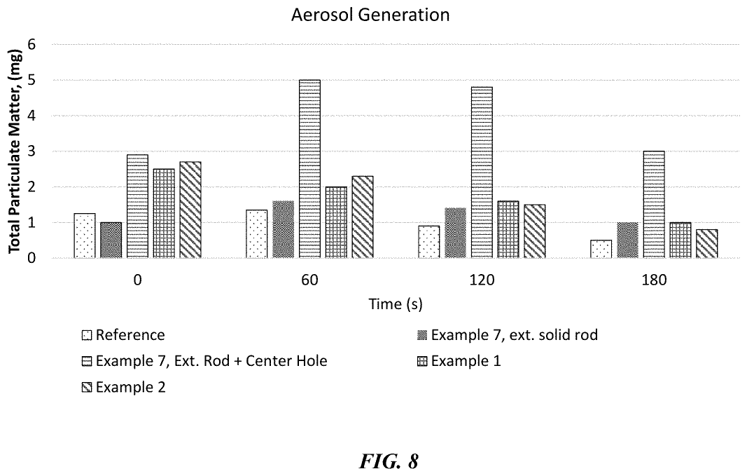

The aerosol formation properties (total particulate matter; TPM) of flat cast sheet substrate embodiments (Examples 1 and 2) and extruded substrate embodiments (Example 7; solid rod and rod with central hole, each with 5 grooves around outer surface) were evaluated in a commercially available heat-not-burn (HNB) device, and compared against the TPM values at various time points for a reference (control) substrate (provided with the commercially available HNB device; Eclipse cast sheet substrate). The control substrate was a cast sheet including tobacco and containing more glycerin compared to the inventive substrates; accordingly, in theory, the control substrate would be capable of volatilizing more aerosol forming material, which would be expected to provide a higher TPM value. TPM in milligrams was determined at 0, 60, 120, and 180 seconds using a 55 ml, 3 second square wave puff. Surprisingly, the results (FIG. 8) demonstrated that the inventive substrates delivered either similar or greater TPM values compared to the control, even at the lower aerosol former (glycerin) loading.

What is claimed is:

1. An aerosol generating component comprising a substrate carrying at least one aerosol forming material, the substrate further comprising:
about 60% dry weight or more of one or more fillers, based on the total dry weight of the substrate, wherein calcium carbonate comprises at least about 80% by dry weight of the one or more fillers; and
at least one binder selected from the group consisting of alginate salts, seaweed hydrocolloids, carrageenans, agar, hydroxyalkyl cellulose ethers, and combinations thereof; and
wherein the substrate is substantially free of solid tobacco material.

2. The aerosol generating component of claim 1, wherein the substrate comprises at least about 70% by dry weight of the one or more fillers, based on the total dry weight of the substrate.

3. The aerosol generating component of claim 1, wherein the substrate comprises at least about 75% by dry weight of the one or more fillers, based on the total dry weight of the substrate.

4. The aerosol generating component of claim 1, wherein the substrate comprises at least about 55% by dry weight of calcium carbonate, based on the total dry weight of the substrate.

5. The aerosol generating component of claim 1, wherein the substrate comprises at least about 60% by dry weight calcium carbonate, based on the total dry weight of the substrate.

6. The aerosol generating component of claim 1, wherein the substrate is substantially free of rice starch and rice flour.

7. The aerosol generating component of claim 1, wherein the substrate is substantially free of wood fibers.

8. The aerosol generating component of claim 1, further comprising a filler selected from the group consisting of native or modified starches, maltodextrin, dextrose, calcium phosphate, lactose, sugar alcohols, microcrystalline cellulose, and combinations thereof.

9. The aerosol generating component of claim 1, wherein the at least one binder is selected from the group consisting of alginate salts, seaweed hydrocolloids, carrageenans, agar, and combinations thereof.

10. The aerosol generating component of claim 1, wherein the at least one binder is a hydroxyalkyl cellulose ether selected from the group consisting of methylcellulose, hydroxypropyl cellulose (HPC), hydroxypropyl methylcellulose (HPMC), hydroxyethyl cellulose, and combinations thereof.

11. The aerosol generating component of claim 1, wherein the at least one binder is selected from the group consisting of hydroxypropyl cellulose (HPC), hydroxypropyl methylcellulose (HPMC), and combinations thereof.

12. The aerosol generating component of claim 1, wherein the at least one binder is a combination of HPC and HPMC.

13. The aerosol generating component of claim 12, wherein a weight ratio of HPC to HPMC is at least about 1:1, such as about 1:1 to about 5:1, about 2:1 to about 4:1, or about 3:1.

14. The aerosol generating component of claim 1, wherein the at least one aerosol forming material comprises water, a polyhydric alcohol, a polysorbate, a sorbitan ester, a fatty acid, a fatty acid ester, a wax, a cannabinoid, a terpene, a sugar alcohol, or a combination of any thereof.

15. The aerosol generating component of claim 14, wherein the at least one aerosol forming material comprises a polyhydric alcohol.

16. The aerosol generating component of claim 15, wherein the polyhydric alcohol is selected from the group consisting of glycerol, propylene glycol, 1,3-propanediol, diethylene glycol, triethylene glycol, triacetin, and combinations thereof.

17. The aerosol generating component of claim 1, wherein the substrate carries the at least one aerosol forming material at a loading of from about 15 to about 55% by weight, based on the total weight of the substrate and aerosol forming material.

18. The aerosol generating component of claim 1, wherein the substrate further carries a flavorant, an active ingredient, a tobacco extract, or a combination thereof.

19. The aerosol generating component of claim 18, wherein the active ingredient comprises a nicotine component.

20. The aerosol generating component of claim 1, wherein the substrate is in particulate form, shredded form, film form, paper process sheet form, cast sheet form, bead form, granular rod form, or extrudate form.

21. The aerosol generating component of claim 20, wherein the substrate is formed into a substantially cylindrical shape, the substrate optionally including at least one of an orifice extending through the substantially cylindrical shape and one or more grooves in an exterior surface thereof.

22. An aerosol generating component comprising a substrate carrying at least one aerosol forming material, the substrate comprising:
about 60% dry weight or more of one or more fillers, based on the total dry weight of the substrate; and
at least one binder, the at least one binder comprising a combination of hydroxypropyl cellulose (HPC) and hydroxypropyl methylcellulose (HPMC);
wherein the substrate is substantially free of solid tobacco material.

23. The aerosol generating component of claim 22, wherein a weight ratio of HPC to HPMC is at least about 1:1, such as about 1:1 to about 5:1 or about 2:1 to about 4:1 or about 3:1.

24. The aerosol generating component of claim 22, wherein the one or more fillers comprises native or modified starches, maltodextrin, dextrose, calcium phosphate, lactose, sugar alcohols, microcrystalline cellulose, or a combination thereof.

25. An aerosol delivery device, comprising:
the aerosol generating component of claim 1;
a heat source configured to heat the substrate carrying the one or more aerosol forming materials to form an aerosol; and
an aerosol pathway extending from the aerosol generating component to a mouth-end of the aerosol delivery device.

26. The aerosol delivery device of claim 25, wherein the heat source comprises either an electrically powered heating element or a combustible ignition source.

27. The aerosol delivery device of claim 26, wherein the heat source is a combustible ignition source comprising a carbon-based material.

28. The aerosol delivery device of claim 26, wherein the heat source is an electrically-powered heating element.

29. The aerosol delivery device of claim 28, further comprising a power source electronically connected to the heating element.

30. The aerosol delivery device of claim 29, further comprising a controller configured to control the power transmitted by the power source to the heating element.
